SEQUENCE LISTING (48 sequences)

SEQ ID NO: 1 - protein, 529 aa, Artificial Sequence
  LLO protein
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 2 - protein, 529 aa, Artificial Sequence; identical to SEQ ID NO: 1
  N-terminal fragment of an LLO protein

SEQ ID NO: 3 - protein, 416 aa, Artificial Sequence
  LLO fragment
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 4 - protein, 639 aa, Artificial Sequence
  ActA protein
MGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPPIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 5 - protein, 639 aa, Artificial Sequence
  ActA protein
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRPTSEEFSSLNSGDFTDDENSETTEEEIDRLADLRDRGTGKHSRNAGFLPLNPFASSPVPSLSPKVSKISDRALISDITKKTPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 6 - protein, 390 aa, Artificial Sequence
  truncated ActA protein
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 7 - protein, 100 aa, Artificial Sequence
  truncated ActA protein
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKG

SEQ ID NO: 8 - protein, 390 aa, Artificial Sequence; identical to SEQ ID NO: 6
  truncated ActA protein

SEQ ID NO: 9 - protein, 100 aa, Artificial Sequence; identical to SEQ ID NO: 7
  truncated ActA protein

SEQ ID NO: 10 - protein, 93 aa, Artificial Sequence
  truncated ActA protein
ATDSEDSSLNTDEWEEEKTEEQPSEVNTGPRYETAREVSSRDIEELEKSNKVKNTNKADLIAMLKAKAEKGPNNNNNNGEQTGNVAINEEASG

SEQ ID NO: 11 - protein, 200 aa, Artificial Sequence
  truncated ActA protein
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 12 - protein, 303 aa, Artificial Sequence
  truncated ActA protein
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSS

SEQ ID NO: 13 - protein, 370 aa, Artificial Sequence
  truncated ActA protein
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSE

SEQ ID NO: 14 - protein, 226 aa, Artificial Sequence
  truncated ActA fused to hly signal peptide
MKKIMLVFITLILVSLPIAQQTEASRATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 15 - DNA, 1170 bp, Artificial Sequence
  recombinant nucleotide encoding a truncated ActA
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 16 - DNA, 678 bp, Artificial Sequence
  recombinant nucleotide encoding a truncated ActA fused to hly signal peptide
atgaaaaaaataatgctagtttttattacacttatattagttagtctaccaattgcgcaacaaactgaagcatctagagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtccgaataacaataataacaacggtgagcaaacaggaaatgtggctataaatgaagaggcttcaggagtcgaccgaccaactctgcaagtggagcgtcgtcatccaggtctgtcatcggatagcgcagcggaaattaaaaaaagaagaaaagccatagcgtcgtcggatagtgagcttgaaagccttacttatccagataaaccaacaaaagcaaataagagaaaagtggcgaaagagtcagttgtggatgcttctgaaagtgacttagattctagcatgcagtcagcagacgagtctacaccacaacctttaaaagcaaatcaaaaaccatttttccctaaagtatttaaaaaaataaaagatgcggggaaatgggtacgtgataaa

SEQ ID NO: 17 - DNA, 1170 bp, Artificial Sequence; identical to SEQ ID NO: 15
  recombinant nucleotide encoding a truncated ActA protein

SEQ ID NO: 18 - protein, 32 aa, Artificial Sequence
  PEST sequence
KENSISSMAPPASPPASPKTPIEKKHADEIDK

SEQ ID NO: 19 - protein, 14 aa, Artificial Sequence
  PEST sequence
KTEEQPSEVNTGPR

SEQ ID NO: 20 - protein, 28 aa, Artificial Sequence
  PEST sequence
KASVTDTSEGDLDSSMQSADESTPQPLK

SEQ ID NO: 21 - protein, 20 aa, Artificial Sequence
  PEST sequence
KNEEVNASDFPPPPTDEELR

SEQ ID NO: 22 - protein, 33 aa, Artificial Sequence
  PEST sequence
RGGIPTSEEFSSLNSGDFTDDENSETTEEEIDR

SEQ ID NO: 23 - protein, 17 aa, Artificial Sequence
  PEST sequence
KQNTASTETTTTNEQPK

SEQ ID NO: 24 - protein, 17 aa, Artificial Sequence
  PEST sequence
KQNTANTETTTTNEQPK

SEQ ID NO: 25 - protein, 98 aa, Artificial Sequence
  E7 protein
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 26 - protein, 105 aa, Artificial Sequence
  E6 protein
MHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQ

SEQ ID NO: 27 - protein, 158 aa, Artificial Sequence
  E6 protein
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 28 - protein, 158 aa, Artificial Sequence
  E6 protein
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 29 - protein, 22 aa, Artificial Sequence
  primer
GGCTCGAGCATGGAGATACACC

SEQ ID NO: 30 - DNA, 28 bp, Artificial Sequence
  Primer
ggggactagtttatggtttctgagaaca

SEQ ID NO: 31 - DNA, 31 bp, Artificial Sequence
  Primer
gggggctagccctcctttgattagtatattc

SEQ ID NO: 32 - DNA, 28 bp, Artificial Sequence
  Primer
ctccctcgagatcataatttacttcatc

SEQ ID NO: 33 - DNA, 55 bp, Artificial Sequence
  Primer
gactacaaggacgatgaccgacaagtgataacccgggatctaaataaatccgttt

SEQ ID NO: 34 - DNA, 27 bp, Artificial Sequence
  Primer
cccgtcgaccagctcttcttggtgaag

SEQ ID NO: 35 - DNA, 25 bp, Artificial Sequence
  Primer
gcggatcccatggagatacacctac

SEQ ID NO: 36 - DNA, 22 bp, Artificial Sequence
  Primer
gctctagattatggtttctgag

SEQ ID NO: 37 - protein, 9 aa, Artificial Sequence
  phycoerythrin (PE)-conjugated E7 peptide
RAHYNIVTF

SEQ ID NO: 38 - protein, 9 aa, Artificial Sequence
  HIV-GAG
AMQMLKETI

SEQ ID NO: 39 - protein, 9 aa, Artificial Sequence
  Listeria LLO
GYKDGNEYI

SEQ ID NO: 40 - protein, 9 aa, Artificial Sequence
  HPV virus gene E7
RAHYNIVTF

SEQ ID NO: 41 - protein, 9 aa, Artificial Sequence
  HIV-Gag peptide
AMQMLKETI

SEQ ID NO: 42 - protein, 9 aa, Artificial Sequence
  LLO 91-99
GYKDGNEYI

SEQ ID NO: 43 - DNA, 20 bp, Artificial Sequence
  human GAPDH -F primer
gccgcatcttcttttgcgtc

SEQ ID NO: 44 - DNA, 20 bp, Artificial Sequence
  human GAPDH -R primer
tcgccccacttgattttgga

SEQ ID NO: 45 - DNA, 20 bp, Artificial Sequence
  human IFN-G -F primer
atggttgtcctgcctgcaat

SEQ ID NO: 46 - DNA, 20 bp, Artificial Sequence
  human IFN-G -R primer
cttgcttaggttggctgcct

SEQ ID NO: 47 - DNA, 20 bp, Artificial Sequence
  human IL-8 -F primer
agtccttgttccactgtgcc

SEQ ID NO: 48 - DNA, 20 bp, Artificial Sequence
  human  IL-8 -R primer
cacagcactaccaacacagc

(19) United States
(12) Patent Application Publication
(10) Pub. No.: US 2016/0220652 A1
(43) Pub. Date: Aug. 4, 2016

(54) METHODS OF USING RECOMBINANT LISTERIA VACCINE STRAINS IN DISEASE IMMUNOTHERAPY

(71) Applicant: Advaxis, Inc., Princeton, NJ (US)

(72) Inventors: Robert PETIT, Newtown (Wrightstown), PA (US); Poonam Molli, North Brunswick, NJ (US)

(73) Assignee: Advaxis, Inc., Princeton, NJ (US)

(21) Appl. No.: 15/014,961

(22) Filed: Feb. 3, 2016

Related U.S. Application Data
(60) Provisional application No. 62/111,210, filed on Feb. 3, 2015.

(51) Int. Cl. A61K 39/02; A61K 45/06

(52) U.S. Cl. CPC A61K 39/0208; A61K 45/06; A61K 2039/522

(57) ABSTRACT

The present disclosure provides methods of treating, protecting against, enhancing and inducing an immune response against a tumor or cancer, comprising the step of administering to a subject a recombinant Listeria. In other embodiments, the Listeria stimulates the STING pathway.

### CROSS-REFERENCE TO RELATED APPLICATIONS

This application claims priority of U.S. Provisional Patent Application No. 62/111,210, filed Feb. 3, 2015, which is hereby incorporated by reference.

### FIELD OF INTEREST

The present disclosure provides methods of treating, protecting against, enhancing and inducing an immune response against a tumor or cancer, comprising the step of administering to a subject a recombinant Listeria. In other embodiments, the Listeria stimulates the STING pathway.

### BACKGROUND

Earlier studies have identified, STING (stimulator of interferon genes), as an essential molecule for cytosolic DNA-mediated type I IFNs induction.

STING, is a crucial part of immune response that allows a host to detect threats—such as infections or cancer cells—that are marked by the presence of DNA that is damaged or in the wrong place, such as in the cytoplasm, outside the nucleus. Detection of cytosolic DNA initiates a series of interactions that lead to the STING pathway. Activating the pathway triggers the production of interferon-beta, which in turn alerts the immune system to the threat, helps the system detect cancerous or infected cells, and ultimately sends activated T cells to respond. Further, the STING pathway when activated triggers a natural immune response against a tumor. This includes production of chemical signals that help the immune system identify tumor cells and generate specific killer T cells.

STING mediates the type I interferon production in response to intracellular DNA and a variety of intracellular pathogens, including viruses, intracellular bacteria and intracellular parasites. Upon infection, STING from infected cells can sense the presence of nucleic acids from intracellular pathogens, and then induce interferon β and more than 10 forms of interferon α production. Type I interferon produced by infected cells can find and bind to Interferon-alpha/beta receptor of nearby cells to protect cells from local infection. Overexpression of STING induces activation of both NF-κB and IRF3 to stimulate type I IFN production.

Listeria monocytogenes (Lm) is a food-borne gram-positive bacterium that can occasionally cause disease in humans, in particular elderly individuals, newborns, pregnant women and immunocompromised individuals. In addition to strongly activating innate immunity and inducing a cytokine response that enhances antigen-presenting cell (APC) function, Lm has the ability to replicate in the cytosol of APCs after escaping from the phagolysosome, mainly through the action of the listeriolysin O (LLO) protein. This unique intracellular life cycle allows antigens secreted by Lm to be processed and presented in the context of both MHC class I and II molecules, resulting in potent cytotoxic CD8⁺ and Th1 CD4⁺ T-cell-mediated immune responses. Lm has been extensively investigated as a vector for cancer immunotherapy in pre-clinical models. Immunization of mice with Lm-LLO-E7 induces regression of established tumors expressing E7 and confers long-term protection. The therapeutic efficacy of Lm-LLO-E7 correlates with its ability to induce E7-specific CTLs that infiltrate the tumor site, mature dendritic cells, reduce the number of intratumoral regulatory CD4⁺ CD25⁺ T cells and inhibit tumor angiogenesis.

Lm has also a number of inherent advantages as a vaccine vector. The bacterium grows very efficiently in vitro without special requirements and it lacks LPS, which is a major toxicity factor in gram-negative bacteria, such as Salmonella. Genetically attenuated Lm vectors also offer additional safety as they can be readily eliminated with antibiotics, in case of serious adverse effects and unlike some viral vectors, no integration of genetic material into the host genome occurs.

Inside the Body Lm is rapidly phagocytosized by monocytes and antigen presenting cells. The phagosome binds to a lysosome creating a phago-lysosome that typically has a reduced pH and proteolytic enzymes. Lm has the ability to escape the phago-lysosome through the secretion of a perforin called listeriolysin-O which can form a pore and allow Lm to escape into the cytoplasm.

The current disclosure employs a modification to Lm such that multiple copies of a ds-DNA plasmid are stably inserted into the listeria in order to provide increased stimulation of the STING pathway resulting in increased levels of interferon being produced.

### SUMMARY

In one aspect, the disclosure relates to a method of activating and enhancing a STimulator of INterferon Genes (STING) complex pathway in a host cell in a subject having a tumor or cancer, the method comprising the step of administering to said subject a composition comprising a recombinant Listeria strain capable of expressing a hemolytic LLO protein from a genomic LLO gene, wherein said activation and enhancement of said STING pathway enhances an immune response in said subject, thereby activating and enhancing a STING pathway.

In another aspect, said recombinant nucleic acid is in a double-stranded plasmid that is stably maintained inside said Listeria strain and which may be present in single or multiple copies.

In one aspect, the disclosure relates to a composition comprising a recombinant Listeria strain capable of expressing a hemolytic LLO protein from a genomic LLO gene, for use in activating and enhancing a Stimulator of Interferon Genes (STING) complex pathway in a host cell in a subject having a tumor or cancer, wherein said activation and enhancement of said STING pathway enhances an immune response in said subject.

In another aspect, said recombinant nucleic acid is in a double-stranded plasmid that is stably maintained inside said Listeria strain and which may be present in single or multiple copies.

Other features and advantages of the present disclosure will become apparent from the following detailed description examples and figures. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure, the disclosure of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-B. Lm-E7 and Lm-LLO-E7 use different expression systems to express and secrete E7. Lm-E7 was generated by introducing a gene cassette into the orfZ domain of the L. monocytogenes genome (FIG. 1A). The hly promoter drives expression of the hly signal sequence and the first five amino acids (AA) of LLO followed by HPV-16 E7. FIG. 1B shows Lm-LLO-E7 was generated by transforming the prfA-strain XFL-7 with the plasmid pGG-55. pGG-55 has the hly promoter driving expression of a nonhemolytic fusion of LLO-E7. pGG-55 also contains the prfA gene to select for retention of the plasmid by XFL-7 in vivo.

FIG. 2. Lm-E7 and Lm-LLO-E7 secrete E7. Lm-Gag (lane 1), Lm-E7 (lane 2), Lm-LLO-NP (lane 3), Lm-LLO-E7 (lane 4), XFL-7 (lane 5), and 10403S (lane 6) were grown overnight at 37° C. in Luria-Bertoni broth. Equivalent numbers of bacteria, as determined by OD at 600 nm absorbance, were pelleted and 18 ml of each supernatant was TCA precipitated. E7 expression was analyzed by Western blot. The blot was probed with an anti-E7 mAb, followed by HRP-conjugated anti-mouse (Amersham), then developed using ECL detection reagents.

FIG. 3. Tumor immunotherapeutic efficacy of LLO-E7 fusions. Tumor size in millimeters in mice is shown at 7, 14, 21, 28 and 56 days post tumor-inoculation. Naive mice: open-circles; Lm-LLO-E7: filled circles; Lm-E7: squares; Lm-Gag: open diamonds; and Lm-LLO-NP: filled triangles.

FIG. 4. Splenocytes from Lm-LLO-E7-immunized mice proliferate when exposed to TC-1 cells. C57BL/6 mice were immunized and boosted with Lm-LLO-E7, Lm-E7, or control rLm strains. Splenocytes were harvested 6 days after the boost and plated with irradiated TC-1 cells at the ratios shown. The cells were pulsed with ³H thymidine and harvested. Cpm is defined as (experimental cpm)−(no-TC-1 control).

FIGS. 5A-B. FIG. 5A shows induction of E7-specific IFN-gamma-secreting CD8⁺ T cells in the spleens and the numbers penetrating the tumors, in mice administered TC-1 tumor cells and subsequently administered Lm-E7, Lm-LLO-E7, Lm-ActA-E7, or no vaccine (naive). FIG. 5B Induction and penetration of E7 specific CD8⁺ cells in the spleens and tumors of the mice described for (A).

FIGS. 6A-B. Listeria constructs containing PEST regions induce a higher percentage of E7-specific lymphocytes within the tumor. FIG. 6A shows representative data from 1 experiment. FIG. 6B shows average and SE of data from all 3 experiments.

FIGS. 7A-B. FIG. 7A shows the effect of passaging on bacterial load (virulence) of recombinant Listeria vaccine vectors. Top panel. Lm-Gag. Bottom panel. Lm-LLO-E7. FIG. 7B shows the effect of passaging on bacterial load of recombinant Lm-E7 in the spleen. Average CFU of live bacteria per milliliter of spleen homogenate from four mice is depicted.

FIG. 8 shows induction of antigen-specific CD8⁺ T-cells for HIV-Gag and LLO after administration of passaged Lm-Gag versus unpassaged Lm-Gag. Mice were immunized with 10³ (A, B, E, F) or 10⁵ (C, D, G, H) CFU passaged Listeria vaccine vectors, and antigen-specific T-cells were analyzed. B, D, F, H: unpassaged Listeria vaccine vectors. A-D immune response to MHC class I HIV-Gag peptide. E-H: immune response to an LLO peptide. I: splenocytes from mice immunized with 10⁵ CFU passaged Lm-Gag stimulated with a control peptide from HPV E7.

FIGS. 9A-C. FIG. 9A shows plasmid isolation throughout LB stability study. FIG. 9B shows plasmid isolation throughout TB stability study. FIG. 9C shows quantitation of TB stability study.

FIG. 10 shows numbers of viable bacteria chloramphenicol (CAP)-resistant and CAP-sensitive colony-forming units (CFU) from bacteria grown in LB. Dark bars: CAP⁺; white bars: CAP⁻. The two dark bars and two white bars for each time point represent duplicate samples.

FIG. 11 shows numbers of viable bacteria CAP-resistant and CAP-sensitive CFU from bacteria grown in TB. Dark bars: CAP⁺; white bars: CAP. The two dark bars and two white bars for each time point represent duplicate samples.

FIGS. 12A-D. Bar graph showing changes in expression of IFNβ between 10403s, XFL7 and ADXS11-001 infected THP-1 cells. Glyceraldehyde-3-phosphate dehydrogenase (GAPDH) was used as internal control (FIG. 12C-D). IFNβ was induced at 4 hours post infection only for 10403S and ADXS11-001 and not for XFL7 (FIG. 12A and FIG. 12C). IL8 used as control gene was induced by all three Lm strains at P4 compared to P0 time point (FIG. 12B and FIG. 12D).

### DETAILED DESCRIPTION

The present disclosure provides methods of treating, protecting against, and inducing an immune response against a disease, comprising the step of administering to a subject a composition comprising a recombinant Listeria strain, wherein the Listeria strain activates and enhances a STimulator of INterferon Genes (STING) complex pathway leading to enhancement of an immune response.

In one embodiment, the present disclosure provides a method of activating and enhancing a STING pathway in a host cell in a subject having a tumor or cancer, the method comprising the step of administering to said subject a composition comprising a recombinant Listeria strain capable of expressing a hemolytic LLO protein from a genomic LLO gene, wherein said activation and enhancement of said STING pathway enhances an immune response in said subject, thereby activating and enhancing a STING pathway. In another embodiment, said Listeria strain comprises multiple copies of a recombinant double stranded nucleic acid, said nucleic acid comprising a first open reading frame encoding a recombinant polypeptide comprising an N-terminal fragment of an LLO protein, wherein said recombinant nucleic acid further comprises a second open reading frame encoding a mutant prfA gene or a metabolic enzyme, wherein administering said Listeria induces an anti-tumor or an anti-cancer immune response in said subject. In another embodiment, the N-terminal fragment of LLO is fused to a heterologous antigen or fragment thereof.

In another embodiment, the present disclosure provides a method of activating and enhancing a STING pathway in a host cell in a subject having a tumor or cancer, the method comprising the step of administering to said subject a composition comprising a recombinant Listeria strain capable of expressing a hemolytic LLO protein from a genomic LLO gene, wherein said activation and enhancement of said STING pathway enhances an immune response in said subject, thereby activating and enhancing a STING pathway. In another embodiment, said Listeria comprises a first double-stranded recombinant nucleic acid, said nucleic acid comprising an open reading frame encoding a recombinant polypeptide comprising an N-terminal fragment of an LLO protein, wherein said recombinant Listeria further comprises a second double stranded recombinant nucleic acid molecule comprising an open reading frame encoding a mutant prfA gene or a metabolic enzyme, thereby activating and enhancing a STING pathway. In another embodiment, an N-terminal fragment of LLO is fused to a heterologous antigen or fragment thereof.

In another embodiment, the present disclosure provides a composition comprising a recombinant Listeria strain capable of expressing a hemolytic LLO protein from a genomic LLO gene for use in activating and enhancing a STimulator of INterferon Genes (STING) complex pathway in a host cell in a subject having a tumor or cancer.

In one embodiment, the Stimulator of interferon genes (STING), also known as transmembrane protein 173 (TMEM173) and MPYS/MITA/ERIS is a protein that in humans is encoded by the TMEM173 gene.

STING plays an important role in innate immunity. STING induces type I interferon production when cells are infected with intracellular pathogens, such as viruses, mycobacteria and intracellular parasites. Type I interferon, mediated by STING, protects infected cells and nearby cells from local infection by binding to the same cell that secretes it (autocrine signaling) and nearby cells (paracrine signaling).

STING is encoded by the TMEM173 gene. It works as both a direct cytosolic DNA sensor (CDS) and an adaptor protein in Type I interferon signaling through different molecular mechanisms. It has been shown to activate downstream transcription factors STAT6 and IRF3 through TBK1, which are responsible for antiviral response and innate immune response against intracellular pathogen.

In one embodiment, during infection of host cells by Listeria monocytogenes (Lm), Listeria is taken up by the host cells in the phagocytic vacuole which is quickly lysed by LLO encoded by Listeria's hly gene, after which the Listeria escapes in the cytosol where it replicates. Upon entry into the cytosol Lm secretes cyclic diadenosine monophosphate (c-di-AMP) which activates the innate immune sensor STING leading to the expression of interferons (IFN-alpha and beta) and co-regulated genes.

In one embodiment, providing multiple copies of a double stranded (ds)-DNA plasmid effectively enhances the STING pathway leading to a more pronounced immune response.

In one embodiment, mouse embryonic fibroblasts (MEFs) derived from STING-deficient mice fail to induce type I IFNs in response to infection with Listeria monocytogenes, or transfection of interferon stimulatory DNA, ISD (which comprises double-stranded 45-base-pair oligonucleotides lacking CpG sequences). In another embodiment, STING is identified to be essential for intracellular DNA-mediated activation of type I IFN in macrophages and in conventional dendritic cells (DCs). In another embodiment, Listeria fails to induce type I IFN in DCs lacking STING either.

In one embodiment, “STING” is also known as MITA/MPYS/ERIS. In one embodiment, the STING provided herein is human STING (hSTING).

In one embodiment, the present disclosure provides a method of inducing an anti-tumor or an anti-cancer immune response in a subject, the method comprising the step of administering to said subject a composition comprising a recombinant Listeria strain, wherein said Listeria strain activates and enhances a STING pathway. In another embodiment, said Listeria comprises a mutation in the endogenous dal, dat or actA genes. In another embodiment, said Listeria comprises a mutation in the endogenous dal, dat and actA genes. In one embodiment, the nucleic acid molecule provided herein comprises a first open reading frame encoding recombinant polypeptide comprising a heterologous antigen or fragment thereof. In another embodiment, the recombinant polypeptide further comprises an N-terminal LLO fused to a heterologous antigen. In another embodiment, the nucleic acid molecule provided herein further comprises a second open reading frame encoding a metabolic enzyme. In another embodiment, the metabolic enzyme complements an endogenous gene that is lacking in the chromosome of the recombinant Listeria strain. In another embodiment, the metabolic enzyme encoded by the second open reading frame is an alanine racemase enzyme (dal). In another embodiment, the metabolic enzyme encoded by the second open reading frame is a D-amino acid transferase enzyme (dat). In another embodiment, the Listeria strains provided herein comprise a mutation, a deletion or inactivation in the genomic dal, dat, or actA genes. In another embodiment, the Listeria strains provided herein comprise a mutation, a deletion or inactivation in the genomic dal, dat, and actA genes. In another embodiment, the Listeria lack the genomic dal, dat or actA genes. In another embodiment, the Listeria lack the genomic dal, dat and actA genes.

In another embodiment, administration of the Listeria provided herein or the Listeria-based immunotherapy provided herein is administered in combination with any additional therapy that enhances a STING pathway.

In one embodiment, the present disclosure provides methods for inducing an anti-disease cytotoxic T-cell (CTL) response in a human subject and treating disorders, and symptoms associated with said disease comprising administration of the recombinant Listeria strain. In one embodiment, provided herein is a recombinant Listeria strain, said recombinant Listeria strain comprising a recombinant nucleic acid, said nucleic acid comprising a first open reading frame encoding a recombinant polypeptide comprising a first an N-terminal fragment of an LLO protein fused to a heterologous antigen or fragment thereof, and wherein said recombinant nucleic acid further comprises a second open reading frame encoding a mutant prfA gene. In one embodiment, the mutant prfA gene is one that encodes a point mutation from amino acid D (which also known as “Asp,” “Aspartate” or “Aspartic acid”) to amino acid V (which is also known as “Val,” or “Valine”) at amino acid position 133. In another embodiment, said Listeria comprising said mutant prfA gene activates and enhances a STING pathway.

In another embodiment, the recombinant Listeria is an attenuated Listeria. It will be appreciated that the terms “Attenuation” or “attenuated” may encompass a bacterium, virus, parasite, infectious organism, prion, tumor cell, gene in the infectious organism, and the like, that is modified to reduce toxicity to a host. The host can be a human or animal, or an organ, tissue, or cell. The bacterium, to give a non-limiting example, can be attenuated to reduce binding to a host cell, to reduce spread from one host cell to another host cell, to reduce extracellular growth, or to reduce intracellular growth in a host cell. In one embodiment, attenuation can be assessed by measuring, e.g., an indicum or indicia of toxicity, the LD₅₀, the rate of clearance from an organ, or the competitive index (see, e.g., Auerbuch, et al. (2001) Infect. Immunity 69:5953-5957). Generally, an attenuation results in an increase in the LD₅₀ and/or an increase in the rate of clearance by at least 25%; more generally by at least 50%; most generally by at least 100% (2-fold); normally by at least 5-fold; more normally by at least 10-fold; most normally by at least 50-fold; often by at least 100-fold; more often by at least 500-fold; and most often by at least 1000-fold; usually by at least 5000-fold; more usually by at least 10,000-fold; and most usually by at least 50,000-fold; and most often by at least 100,000-fold. In another embodiment, attenuation results in an increase in the LD₅₀ and/or an increase in the rate of clearance by at least 25%. In another embodiment, attenuation results in an increase in the LD₅₀ and/or an increase in the rate of clearance by 3-5 fold. In other embodiments, attenuation results in an increase in the LD₅₀ and/or an increase in the rate of clearance by 5-10 fold, 11-20 fold, 21-30 fold, 31-40 fold, 41-50 fold, 51-100 fold, 101-500 fold, 501-1,000 fold, 1001-10,000 fold, or 10,001-100,000 fold.

It will be well appreciated by a skilled artisan that the term “Attenuated gene” may encompass a gene that mediates toxicity, pathology, or virulence, to a host, growth within the host, or survival within the host, where the gene is mutated in a way that mitigates, reduces, or eliminates the toxicity, pathology, or virulence. The reduction or elimination can be assessed by comparing the virulence or toxicity mediated by the mutated gene with that mediated by the non-mutated (or parent) gene. “Mutated gene” encompasses deletions, point mutations, inversions, truncations, and frameshift mutations in regulatory regions of the gene, coding regions of the gene, non-coding regions of the gene, or any combination thereof. In one embodiment, the attenuated gene is a gene that is inactivated.

In one embodiment, provided herein is a method for inducing an immune response against a tumor or a cancer in a subject, the method comprising the step of administering to said subject a recombinant Listeria strain comprising a recombinant nucleic acid, said nucleic acid comprising a first open reading frame encoding a recombinant polypeptide comprising an N-terminal fragment of an LLO protein fused to a heterologous antigen or fragment thereof, is, wherein said recombinant nucleic acid further comprises a second open reading frame encoding a mutant prfA gene, thereby inducing an immune response against a tumor or a cancer.

In one embodiment, the present disclosure provides a method of treating a cancer in a subject, comprising the step of administering to the subject the recombinant Listeria strain provided herein. In another embodiment, the present disclosure provides a method of protecting a subject against a cervical cancer, comprising the step of administering to the subject the recombinant Listeria strain provided herein. In another embodiment, the recombinant Listeria strain expresses the recombinant polypeptide. In another embodiment, the recombinant Listeria strain comprises a plasmid that encodes the recombinant polypeptide. In another embodiment, the plasmid is a multi-copy double stranded plasmid. In another embodiment, the method further comprises the step of boosting the subject with a recombinant Listeria strain of the present disclosure. In another embodiment, the method further comprises the step of boosting the subject with an immunogenic composition comprising a heterologous antigen or fragment thereof provided herein. In another embodiment, the method further comprises the step of boosting the subject with an immunogenic composition that directs a cell of the subject to express the heterologous antigen. In another embodiment, the cell is a tumor cell. In another embodiment, the method further comprises the step of boosting the subject with the vaccine of the present disclosure.

In one embodiment, the present disclosure provides a composition comprising a recombinant Listeria strain capable of expressing a hemolytic LLO protein from a genomic LLO gene for use in activating and enhancing a STimulator of INterferon Genes (STING) complex pathway in a host cell in a subject having a tumor or cancer. In another embodiment the activation and enhancement of the STING pathway enhances an immune response in said subject.

In another embodiment, the present disclosure provides a composition comprising a recombinant Listeria strain capable of expressing a hemolytic LLO protein from a genomic LLO gene for use in activating and enhancing a STimulator of INterferon Genes (STING) complex pathway in a host cell in a subject having a tumor or cancer, wherein the Listeria strain comprises multiple copies of a recombinant double stranded nucleic acid, the nucleic acid comprising a first open reading frame encoding a recombinant polypeptide comprising an N-terminal fragment of an LLO protein, wherein the recombinant nucleic acid further comprises a second open reading frame encoding a mutant prfA gene or a metabolic enzyme. In another embodiment, the activation and enhancement of the STING pathway induces an anti-tumor or an anti-cancer immune response in the subject.

In one embodiment, the present disclosure provides a composition comprising a recombinant Listeria strain comprising a recombinant nucleic acid, said nucleic acid comprising a first open reading frame encoding a recombinant polypeptide comprising an N-terminal fragment of an LLO protein fused to a heterologous antigen or fragment thereof, is, wherein said recombinant nucleic acid further comprises a second open reading frame encoding a mutant prfA gene, for use in inducing an immune response against a tumor or a cancer. In another embodiment the present disclosure provides the Listeria strains and compositions, provided herein, for use in activation and enhancement of said STING pathway. In another embodiment the present disclosure provides the Listeria strains and compositions, provided herein, for use in a therapeutic method for activation and enhancement of said STING pathway thereby enhancing an immune response. In another embodiment the present disclosure provides the Listeria strains and compositions provided herein for use as a medicament for treating a tumor or cancer in a subject. In another embodiment the present disclosure provides the Listeria strains and compositions provided herein for use as a medicament for enhancing an immune response in a subject. In another embodiment the present disclosure provides the Listeria strains and compositions provided herein for use as a medicament for activation and enhancement of said STING pathway. In another embodiment the present disclosure provides the Listeria strains and compositions provided herein for use as a medicament for enhancing an immune response in a subject.

In another embodiment, the present disclosure provides the Listeria strains, methods, and compositions, provided herein, for use in activation and enhancement of said STING pathway and inducing an anti-tumor or an anti-cancer immune response in said subject. In another embodiment, the present disclosure provides the Listeria strains, methods, and compositions, provided herein, for use in enhancing production of interferons. In another embodiment, the present disclosure provides the Listeria strains, methods, and compositions, provided herein, for use in enhancing production of interferon beta. In another embodiment, the present disclosure provides the Listeria strains, methods, and compositions, provided herein, for use in leading to a potent anti-tumor cytotoxic T cell response. In another embodiment, the present disclosure provides the Listeria strains, methods, and compositions, provided herein, for use in protecting said subject against a tumor or cancer. In another embodiment, the present disclosure provides the Listeria strains, methods, and compositions, provided herein, for use in induction of an anti-tumor cytotoxic T cell response in a subject. In another embodiment, the present disclosure provides the Listeria strains, methods, and compositions, provided herein, for use in treating a subject having a tumor or cancer. In another embodiment, the present disclosure provides the Listeria strains, methods, and compositions, provided herein, for use in reducing the need of said subject having a tumor or a cancer to receive chemotherapeutic or radiation treatment. In another embodiment, the present disclosure provides the Listeria strains, methods, and compositions, provided herein, for use in reducing the severity of side effects associated with a follow-up radiation or chemotherapeutic treatment in said subject. In another embodiment, the present disclosure provides the Listeria strains, methods, and compositions, provided herein, for use in eliminating the need of a follow-up radiation or chemotherapeutic treatment in said subject having said tumor or cancer. In another embodiment, the present disclosure provides the Listeria strains, methods, and compositions, provided herein, for use in administering a boost dose of said composition comprising said recombinant Listeria strain to said subject. In another embodiment, a composition for use described herein comprises a booster dose of said composition for use at least at a second time point.

In another embodiment, the present disclosure provides the Listeria strains, methods, and compositions, provided herein, for use in protecting a subject against a cervical cancer, comprising the step of administering to the subject the recombinant Listeria strain provided herein. In another embodiment, the recombinant Listeria strain expresses the recombinant polypeptide. In another embodiment, the recombinant Listeria strain comprises a plasmid that encodes the recombinant polypeptide. In another embodiment, the plasmid is a multi-copy double stranded plasmid. In another embodiment, the method further comprises the step of boosting the subject with a recombinant Listeria strain of the present disclosure. In another embodiment, the use further comprises the step of boosting the subject with an immunogenic composition comprising a heterologous antigen or fragment thereof provided herein. In another embodiment, the use further comprises the step of boosting the subject with an immunogenic composition that directs a cell of the subject to express the heterologous antigen. In another embodiment, the cell is a tumor cell. In another embodiment, the use further comprises the step of boosting the subject with the composition of the present disclosure. In another embodiment, a composition for use described herein comprises a booster dose of said composition for use at least at a second time point.

In one embodiment, the fragment thereof in the context of LLO proteins and ActA proteins provided herein refer to a peptide or polypeptide comprising an amino acid sequence of at least 5 contiguous amino acid residues of the LLO or ActA proteins. In another embodiment, the term refers to a peptide or polypeptide comprising an amino acid sequence of at least of at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, at least 250 contiguous amino acid residues of the amino acid sequence, at least 300 contiguous amino acid residues, at least 350 contiguous amino acid residues of, at least 400 contiguous amino acid residues, or at least 450 contiguous amino acid residues of an LLO or ActA protein or polypeptide. In another embodiment, an N-terminal LLO or N-terminal ActA comprise a PEST-sequence. In another embodiment, an N-terminal LLO or an N-terminal ActA are PEST-containing peptides or polypeptides.

In another embodiment, the fragment is a functional fragment that works as intended by the present disclosure (e.g. to elicit an immune response against a disease-associated antigen when in the form of an N-terminal LLO/heterologous antigen fusion protein or N-terminal ActA/heterologous antigen fusion protein). In another embodiment, the fragment is functional in a non-fused form.

The N-terminal LLO fragment or N-terminal ActAs protein fragment and heterologous antigen are, in another embodiment, fused directly to one another. In another embodiment, the genes encoding the N-terminal LLO protein fragment or the ActA protein fragment and the heterologous antigen are fused directly to one another. In another embodiment, the N-terminal LLO protein fragment or the ActA protein fragment and the heterologous antigen are attached via a linker peptide. In another embodiment, the N-terminal LLO protein fragment or the ActA protein fragment and the heterologous antigen are attached via a heterologous peptide. In another embodiment, the N-terminal LLO protein fragment or the ActA protein fragment is N-terminal to the heterologous antigen. In another embodiment, the N-terminal LLO protein fragment or the ActA protein fragment is the N-terminal-most portion of the fusion protein. Each possibility represents a separate embodiment of the present disclosure.

As provided herein, recombinant Listeria strains expressing LLO-antigen fusions induce anti-tumor immunity (Example 1), elicit antigen-specific T cell proliferation (Example 2), generate antigen-specific, and tumor-infiltrating T cells (Example 3).

In another embodiment, the present disclosure provides a method of treating a solid cancer or tumor in said subject. In another embodiment, the subject is a human subject. In another embodiment, the present disclosure provides a method of treating a solid tumor or cancer in a human subject, comprising the step of administering to the subject a recombinant Listeria strain, the recombinant Listeria strain comprising a recombinant polypeptide comprising an N-terminal fragment of an LLO or ActA protein and an antigen associated with said solid cancer or tumor. In another embodiment, the recombinant Listeria strain expresses the recombinant polypeptide. In another embodiment, the recombinant Listeria strain comprises a plasmid that encodes the recombinant polypeptide. In another embodiment, the plasmid is a multi-copy plasmid.

In one embodiment, the present disclosure provides a method for vaccinating a subject against a tumor or cancer, comprising the step of administering to the subject a recombinant Listeria strain provided herein or an immunogenic composition comprising the same. In another embodiment, the subject is a human subject. In another embodiment, the subject is a human child. In another embodiment, the subject is a non-human mammal.

In one embodiment, the Listeria expresses a tumor associated antigen. In another embodiment, a tumor-associated antigen is a naturally occurring tumor-associated antigen. In another embodiment, the tumor-associated antigen is a synthetic tumor-associated antigen. In yet another embodiment, the tumor-associated antigen is a chimeric tumor-associated antigen. In another embodiment, a tumor-associated antigen is a heterologous antigen. In another embodiment, a tumor-associated antigen is a self-antigen. In another embodiment, a tumor associated antigen is an angiogenic antigen.

It will be appreciated by the skilled artisan that an “antigen” or “antigenic polypeptide” may encompass a polypeptide, peptide or recombinant peptide as described herein that is processed and presented on MHC class I and/or class II molecules present in a subject's cells leading to the mounting of an immune response when present in, or in another embodiment, detected by, the host. In one embodiment, the antigen may be foreign to the host. In another embodiment, the antigen might be present in the host but the host does not elicit an immune response against it because of immunologic tolerance.

In one embodiment, the methods provided herein overcome or break tolerance to self. In another embodiment, the present disclosure provides the Listeria strains, methods, and compositions, provided herein, for use in breaking tolerance to self.

In one embodiment, the nucleic acid molecule provided herein is used to transform the Listeria in order to arrive at a recombinant Listeria. In another embodiment, the nucleic acid provided herein used to transform a Listeria that lacks a virulence gene. In another embodiment, the nucleic acid molecule is integrated into the Listeria genome that carries a non-functional virulence gene. In another embodiment, the Listeria comprises a mutation in a virulence gene. In yet another embodiment, the nucleic acid molecule is used to inactivate a gene (e.g. metabolic, virulence gene, or any other gene) present in the Listeria genome. In another embodiment, the Listeria already comprises an inactivation of a virulence gene. In yet another embodiment, the virulence gene provided herein is an actA gene, an inlA gene, an inlB gene, an inlC gene or a prfA gene. In one embodiment, the Listeria is a double mutant. In another embodiment, the Listeria is an actA/inlB double mutant. As will be understood by a skilled artisan, the virulence gene can be any gene known in the art to be associated with virulence in the recombinant Listeria.

In one embodiment, a mutant Listeria comprising a mutant genomic prfA gene is complemented by use of a plasmid that expresses a mutant prfA gene. In another embodiment, the mutant prfA gene is a D133V prfA mutation.

In one embodiment, the recombinant Listeria strain comprises a bivalent episomal expression vector, the vector comprising a first, and a second nucleic acid molecule encoding a heterologous antigenic polypeptide or a functional fragment thereof, wherein the first and the second nucleic acid molecules each encode the heterologous antigenic polypeptide or functional fragment thereof in an open reading frame with an endogenous PEST-containing polypeptide or peptide. In another embodiment, a PEST-containing polypeptide is a N-terminal or truncated or detoxified Listeriolysin O protein (LLO). In another embodiment, the PEST-containing polypeptide is an N-terminal or a truncated ActA protein. In another embodiment, the PEST-containing peptide is a PEST amino acid sequence. In another embodiment, such a Listeria comprising a bivalent episomal vector is more efficient at stimulating a STING pathway due to the higher presence of ds-DNA in the form of a first and second nucleic acid molecules.

In one embodiment, the heterologous antigens expressed by bivalent expression vector are any of the antigens provided herein and known in the art.

In one embodiment, the recombinant Listeria is trivalent in that it expresses three heterologous antigens or functional fragments thereof.

In one embodiment, provided herein is a trivalent, recombinant Listeria strain expressing three heterologous antigens individually fused to a PEST-containing polypeptide or peptide provided herein.

In one embodiment, provided herein is a quadravalent recombinant Listeria strain expressing four heterologous antigens individually fused to a PEST-containing polypeptide or peptide provided herein.

In one embodiment, the bivalent, trivalent, or quadravalent recombinant Listeria strains provided herein express at least one heterologous antigen from an open reading frame in a extrachromosomal plasmid or episome. In another embodiment, the bivalent, trivalent, or quadravalent recombinant Listeria strains provided herein express at least one heterologous antigen from an open reading frame from at least one extrachromosomal plasmid or episome. In another embodiment, the bivalent recombinant Listeria strains provided herein express two heterologous antigens each from an open reading frame of two extrachromosomal plasmids or episomes. In another embodiment, the trivalent recombinant Listeria strains provided herein express three heterologous antigens each from an open reading frame of three extrachromosomal plasmids or episomes. In another embodiment, the quadravalent recombinant Listeria strains provided herein express four heterologous antigens each from an open reading frame of four extrachromosomal plasmids or episomes. In another embodiment, the higher the valency of expressed heterologous antigens, the higher the level of stimulation of a STING pathway that the Listeria can stimulate in a host cell.

In another embodiment, the bivalent, trivalent, or quadravalent recombinant Listeria strains provided herein express at least one heterologous antigen from an open reading frame in the genome of the Listeria. In another embodiment, the bivalent, trivalent, or quadravalent recombinant Listeria strains provided herein express at least one heterologous antigen from both, an extrachromosomal plasmid or episome, and from the genome of a Listeria provided herein. In another embodiment, each heterologous antigen is expressed in a fusion protein with a PEST-containing polypeptide or peptide provided herein. In another embodiment, the higher the valency of expressed heterologous antigens in a fusion protein with a PEST-containing polypeptide or peptide, the higher the level of stimulation of a STING pathway that the Listeria can stimulate in a host cell.

In one embodiment, bivalent and multivalent recombinant Listeria encompassed by the present disclosure includes those described in US Pub. No. 2011/0129499, and in US Pub No. 2012/0135033, both of which are incorporated by reference in their entirety herein. These Listeria strains are also envisioned in the compositions and methods provided herein.

In another embodiment, the present disclosure provides a method of inducing a cytotoxic T cell (CTL) response in a human subject against an antigen of interest, the method comprising the step of administering to the human subject a recombinant Listeria strain comprising or expressing the antigen of interest, thereby inducing a CTL response in a human subject against an antigen of interest. In another embodiment, an antigen of interest is a heterologous antigen, which in another embodiment is a tumor associated antigen.

In another embodiment, the present disclosure provides a method for inducing a regression of a cancer in a subject, comprising the step of administering to the subject a composition comprising the recombinant Listeria strain provided herein

In another embodiment, the present disclosure provides a method for reducing an incidence of relapse of a cancer in a subject, comprising the step of administering to the subject a composition comprising the recombinant Listeria strain provided herein.

In another embodiment, the present disclosure provides a method for suppressing a formation of a tumor in a subject, comprising the step of administering to the subject a composition comprising the recombinant Listeria strain provided herein.

In another embodiment, the present disclosure provides a method for inducing a remission of a cancer in a subject, comprising the step of administering to the subject a composition comprising the recombinant Listeria strain provided herein.

In another embodiment, the present disclosure provides a method for impeding a growth of a tumor in a human subject, comprising the step of administering to the subject a composition comprising the recombinant Listeria strain provided herein.

In another embodiment, the present disclosure provides a method for reducing a size of a tumor in a subject, comprising the step of administering to the subject the recombinant Listeria strain provided herein.

In another embodiment, the present invention disclosure provides the Listeria strains, compositions, and methods provided herein, for use in inducing a cytotoxic T cell (CTL) response in a human subject against an antigen of interest, the use comprising administering to the human subject a recombinant Listeria strain comprising or expressing the antigen of interest, thereby inducing a CTL response in a human subject against an antigen of interest. In another embodiment, an antigen of interest is a heterologous antigen, which in another embodiment is a tumor associated antigen.

In another embodiment, the present invention disclosure provides the Listeria strains, compositions, and methods, provided herein, for use in inducing a regression of a cancer in a subject, comprising administering to the subject a composition comprising the recombinant Listeria strain provided herein.

In another embodiment, the present invention disclosure provides the Listeria strains, compositions, and methods, provided herein, for use in reducing an incidence of relapse of a cancer in a subject, comprising administering to the subject a composition comprising the recombinant Listeria strain provided herein.

In another embodiment, the present invention disclosure provides the Listeria strains, compositions, and methods, provided herein, for use in suppressing a formation of a tumor in a subject, comprising administering to the subject a composition comprising the recombinant Listeria strain provided herein.

In another embodiment, the present invention disclosure provides the Listeria strains, compositions, and methods, provided herein, for use in inducing a remission of a cancer in a subject, comprising administering to the subject a composition comprising the recombinant Listeria strain provided herein.

In another embodiment, the present invention disclosure provides the Listeria strains, compositions, and methods, provided herein, for use in impeding a growth of a tumor in a human subject, comprising administering to the subject a composition comprising the recombinant Listeria strain provided herein.

In another embodiment, the present invention disclosure provides the Listeria strains, compositions, and methods, provided herein, for use in reducing a size of a tumor in a subject, comprising administering to the subject the recombinant Listeria strain provided herein.

In another embodiment, the present invention disclosure provides the Listeria strains, compositions, and methods, provided herein, for use in treating a disease.

In one embodiment, the disease is an infectious disease, an autoimmune disease, a respiratory disease, a pre-cancerous condition or a cancer.

It will be well appreciated by the skilled artisan that the term “pre-cancerous condition” may encompass dysplasias, preneoplastic nodules; macroregenerative nodules (MRN); low-grade dysplastic nodules (LG-DN); high-grade dysplastic nodules (HG-DN); biliary epithelial dysplasia; foci of altered hepatocytes (FAH); nodules of altered hepatocytes (NAH); chromosomal imbalances; aberrant activation of telomerase; re-expression of the catalytic subunit of telomerase; expression of endothelial cell markers such as CD31, CD34, and BNH9 (see, e.g., Terracciano and Tomillo (2003) Pathologica 95:71-82; Su and Bannasch (2003) Toxicol. Pathol. 31:126-133; Rocken and Carl-McGrath (2001) Dig. Dis. 19:269-278; Kotoula, et al. (2002) Liver 22:57-69; Frachon, et al. (2001) J. Hepatol. 34:850-857; Shimonishi, et al. (2000) J. Hepatobiliary Pancreat. Surg. 7:542-550; Nakanuma, et al. (2003) J. Hepatobiliary Pancreat. Surg. 10:265-281). Methods for diagnosing cancer and dysplasia are disclosed (see, e.g., Riegler (1996) Semin. Gastrointest. Dis. 7:74-87; Benvegnu, et al. (1992) Liver 12:80-83; Giannini, et al. (1987) Hepatogastroenterol. 34:95-97; Anthony (1976) Cancer Res. 36:2579-2583).

In one embodiment, an infectious disease is one caused by, but not limited to, any one of the following pathogens: BCG/Tuberculosis, Malaria, Plasmodium falciparum, plasmodium malariae, plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Vaccines, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Trichomonas vaginalis, or any other infectious disease known in the art that is not listed herein.

In another embodiment, the infectious disease is a livestock infectious disease. In another embodiment, livestock diseases can be transmitted to man and are called “zoonotic diseases.” In another embodiment, these diseases include, but are not limited to, Foot and mouth disease, West Nile Virus, rabies, canine parvovirus, feline leukemia virus, equine influenza virus, infectious bovine rhinotracheitis (IBR), pseudorabies, classical swine fever (CSF), IBR, caused by bovine herpesvirus type 1 (BHV-1) infection of cattle, and pseudorabies (Aujeszky's disease) in pigs, toxoplasmosis, anthrax, vesicular stomatitis virus, rhodococcus equi, Tularemia, Plague (Yersinia pestis), trichomonas.

In another embodiment, the disease provided herein is a respiratory or inflammatory disease. In another embodiment, the respiratory or inflammatory disease is chronic obstructive pulmonary disease (COPD). In another embodiment, the disease is asthma.

In one embodiment, live attenuated Listeria strains are capable of alleviating asthma symptoms without co-administration of other therapeutic agents, such as anti-inflammatory agents or bronchodilators. In another embodiment, the methods provided herein further comprise the step of co-administering to a subject the live attenuated Listeria strain and one or more therapeutic agents. In another embodiment, the therapeutic agent is an anti-asthmatic agent. In another embodiment, the agent is an anti-inflammatory agent, a non-steroidal anti-inflammatory agent, an antibiotic, an antichlolinerginc agent, a bronchodilator, a corticosteroid, a short-acting beta-agonist, a long-acting beta-agonist, combination inhalers, an antihistamine, or combinations thereof.

In one embodiment, the disease provided herein is a cancer or a tumor. In one embodiment, the tumor is cancerous. In another embodiment, the cancer is breast cancer. In another embodiment, the cancer is a cervical cancer. In another embodiment, the cancer is a Her2 containing cancer. In another embodiment, the cancer is a melanoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, it is a glioblastoma multiforme. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non-small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma. In another embodiment, the cancer is oropharyngeal cancer. In another embodiment, the cancer is lung cancer. In another embodiment, the cancer is anal cancer. In another embodiment, the cancer is colorectal cancer. In another embodiment, the cancer is esophageal cancer. The cervical tumor targeted by methods and uses of the present disclosure is, in another embodiment, a squamous cell carcinoma. In another embodiment, the cervical tumor is an adenocarcinoma. In another embodiment, the cervical tumor is an adenosquamous carcinoma. In another embodiment, the cervical tumor is a small cell carcinoma. In another embodiment, the cervical tumor is any other type of cervical tumor known in the art.

The cervical tumor targeted by methods and uses of the present disclosure is, in another embodiment, a squamous cell carcinoma. In another embodiment, the cervical tumor is an adenocarcinoma. In another embodiment, the cervical tumor is an adenosquamous carcinoma. In another embodiment, the cervical tumor is a small cell carcinoma. In another embodiment, the cervical tumor is any other type of cervical tumor known in the art.

In one embodiment, the antigen provided herein is a heterologous tumor antigen, which is also referred to herein as “tumor antigen” “antigenic polypeptide,” or “foreign antigen.” In another embodiment, the antigen is Human Papilloma Virus-E7 (HPV-E7) antigen, which in one embodiment, is from HPV16 (in one embodiment, GenBank Accession No. AAD33253) and in another embodiment, from HPV18 (in one embodiment, GenBank Accession No. P06788). In another embodiment, the antigenic polypeptide is HPV-E6, which in one embodiment, is from HPV16 (in one embodiment, GenBank Accession No. AAD33252, AAM51854, AAM51853, or AAB67615) and in another embodiment, from HPV18 (in one embodiment, GenBank Accession No. P06463). In another embodiment, the antigenic polypeptide is a Her/2-neu antigen. In another embodiment, the antigenic polypeptide is Prostate Specific Antigen (PSA) (in one embodiment, GenBank Accession No. CAD30844, CAD54617, AAA58802, or NP_001639). In another embodiment, the antigenic polypeptide is Stratum Corneum Chymotryptic Enzyme (SCCE) antigen (in one embodiment, GenBank Accession No. AAK69652, AAK69624, AAG33360, AAF01139, or AAC37551). In another embodiment, the antigenic polypeptide is Wilms tumor antigen 1, which in another embodiment is WT-1 Telomerase (GenBank Accession. No. P49952, P22561, NP_659032, CAC39220.2, or EAW68222.1). In another embodiment, the antigenic polypeptide is hTERT or Telomerase (GenBank Accession. No. NM003219 (variant 1), NM198255 (variant 2), NM 198253 (variant 3), or NM 198254 (variant 4). In another embodiment, the antigenic polypeptide is Proteinase 3 (in one embodiment, GenBank Accession No. M29142, M75154, M96839, X55668, NM 00277, M96628 or X56606). In another embodiment, the antigenic polypeptide is Tyrosinase Related Protein 2 (TRP2) (in one embodiment, GenBank Accession No. NP_001913, ABI73976, AAP33051, or Q95119). In another embodiment, the antigenic polypeptide is High Molecular Weight Melanoma Associated Antigen (HMW-MAA) (in one embodiment, GenBank Accession No. NP_001888, AAI28111, or AAQ62842). In another embodiment, the antigenic polypeptide is Testisin (in one embodiment, GenBank Accession No. AAF79020, AAF79019, AAG02255, AAK29360, AAD41588, or NP_659206). In another embodiment, the antigenic polypeptide is NY-ESO-1 antigen (in one embodiment, GenBank Accession No. CAA05908, P78358, AAB49693, or NP_640343). In another embodiment, the antigenic polypeptide is PSCA (in one embodiment, GenBank Accession No. AAH65183, NP_005663, NP_082492, 043653, or CAB97347). In another embodiment, the antigenic polypeptide is Interleukin (IL) 13 Receptor alpha (in one embodiment, GenBank Accession No. NP_000631, NP_001551, NP_032382, NP_598751, NP_001003075, or NP_999506). In another embodiment, the antigenic polypeptide is Carbonic anhydrase IX (CAIX) (in one embodiment, GenBank Accession No. CAI13455, CAI10985, EAW58359, NP_001207, NP_647466, or NP_001101426). In another embodiment, the antigenic polypeptide is carcinoembryonic antigen (CEA) (in one embodiment, GenBank Accession No. AAA66186, CAA79884, CAA66955, AAA51966, AAD15250, or AAA51970.). In another embodiment, the antigenic polypeptide is MAGE-A (in one embodiment, GenBank Accession No. NP_786885, NP_786884, NP_005352, NP_004979, NP_005358, or NP_005353). In another embodiment, the antigenic polypeptide is survivin (in one embodiment, GenBank Accession No. AAC51660, AAY15202, ABF60110, NP_001003019, or NP_001082350). In another embodiment, the antigenic polypeptide is GP100 (in one embodiment, GenBank Accession No. AAC60634, YP_655861, or AAB31176). In another embodiment, the antigenic polypeptide is any other antigenic polypeptide known in the art. In another embodiment, the antigenic peptide of the compositions and methods of the present disclosure comprise an immunogenic portion of the antigenic polypeptide.

In another embodiment, the antigen is HPV-E6. In another embodiment, the antigen is telomerase (TERT). In another embodiment, the antigen is LMP-1. In another embodiment, the antigen is p53. In another embodiment, the antigen is mesothelin. In another embodiment, the antigen is EGFRVIII. In another embodiment, the antigen is carboxic anhydrase IX (CAIX). In another embodiment, the antigen is PSMA. In another embodiment, the antigen is HMW-MAA. In another embodiment, the antigen is HIV-1 Gag. In another embodiment, the antigen is Tyrosinase related protein 2. In another embodiment, the antigen is selected from HPV-E7, HPV-E6, Her-2, HIV-1 Gag, LMP-1, p53, PSMA, carcinoembryonic antigen (CEA), LMP-1, kallikrein-related peptidase 3 (KLK3), KLK9, Muc, Tyrosinase related protein 2, Muc1, FAP, IL-13R alpha 2, PSA (prostate-specific antigen), gp-100, heat-shock protein 70 (HSP-70), beta-HCG, EGFR-III, Granulocyte colony-stimulating factor (G-CSF), Angiogenin, Angiopoietin-1, Del-1, Fibroblast growth factors: acidic (aFGF) or basic (bFGF), Follistatin, Granulocyte colony-stimulating factor (G-CSF), Hepatocyte growth factor (HGF)/scatter factor (SF), Interleukin-8 (IL-8), Leptin, Midkine, Placental growth factor, Platelet-derived endothelial cell growth factor (PD-ECGF), Platelet-derived growth factor-BB (PDGF-BB), Pleiotrophin (PTN), Progranulin, Proliferin, Transforming growth factor-alpha (TGF-alpha), Transforming growth factor-beta (TGF-beta), Tumor necrosis factor-alpha (TNF-alpha), Vascular endothelial growth factor (VEGF)/vascular permeability factor (VPF), VEGFR, VEGFR2 (KDR/FLK-1) or a fragment thereof, FLK-1 or an epitope thereof, FLK-E1, FLK-E2, FLK-I1, endoglin or a fragment thereof, Neuropilin 1 (NRP-1), Angiopoietin 1 (Ang1), Tie2, Platelet-derived growth factor (PDGF), Platelet-derived growth factor receptor (PDGFR), Transforming growth factor-beta (TGF-β), endoglin, TGF-β receptors, monocyte chemotactic protein-1 (MCP-1), VE-cadherin, CD31, ephrin, ICAM-1, V-CAM-1, VAP-1, E-selectin, plasminogen activators, plasminogen activator inhibitor-1, Nitric oxide synthase (NOS), COX-2, AC133, or Id1/Id3, Angiopoietin 3, Angiopoietin 4, Angiopoietin 6, CD105, EDG, HHT1, ORW, ORW1 or a TGFbeta co-receptor, or a combination thereof. In another embodiment, the antigen is a chimeric Her2/neu antigen as disclosed in US Patent Application Publication No. 2011/0142791, which is incorporated by reference herein in its entirety. The use of fragments of antigens provided herein is also encompassed by the present disclosure.

In another embodiment, the heterologous tumor antigen provided herein is a tumor-associated antigen, which in one embodiment, is one of the following tumor antigens: a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, a MART1 antigen associated with melanoma, or the PSA antigen associated with prostate cancer. In another embodiment, the antigen for the compositions and methods provided herein are melanoma-associated antigens, which in one embodiment are TRP-2, MAGE-1, MAGE-3, gp-100, tyrosinase, HSP-70, beta-HCG, or a combination thereof. It is to be understood that a skilled artisan would be able to use any heterologous antigen not mentioned herein but known in the art for use in the methods and compositions provided herein. It is also to be understood that the present disclosure provides, but is not limited by, an attenuated Listeria comprising a nucleic acid that encodes at least one of the antigens disclosed herein. The present disclosure encompasses nucleic acids encoding mutants, muteins, splice variants, fragments, truncated variants, soluble variants, extracellular domains, intracellular domains, mature sequences, and the like, of the disclosed antigens. Provided are nucleic acids encoding epitopes, oligo- and polypeptides of these antigens. Also provided are codon optimized embodiments, that is, optimized for expression in Listeria. The cited references, GenBank Acc. Nos., and the nucleic acids, peptides, and polypeptides disclosed herein, are all incorporated herein by reference in their entirety. In another embodiment, the selected nucleic acid sequence can encode a full length or a truncated gene, a fusion or tagged gene, and can be a cDNA, a genomic DNA, or a DNA fragment, preferably, a cDNA. It can be mutated or otherwise modified as desired. These modifications include codon optimizations to optimize codon usage in the selected host cell or bacteria, i.e. Listeria. The selected sequence can also encode a secreted, cytoplasmic, nuclear, membrane bound or cell surface polypeptide.

In one embodiment, vascular endothelial growth factor (VEGF) is an important signaling protein involved in both vasculogenesis (the formation of the embryonic circulatory system) and angiogenesis (the growth of blood vessels from pre-existing vasculature). In one embodiment, VEGF activity is restricted mainly to cells of the vascular endothelium, although it does have effects on a limited number of other cell types (e.g. stimulation monocyte/macrophage migration). In vitro, VEGF has been shown to stimulate endothelial cell mitogenesis and cell migration. VEGF also enhances microvascular permeability and is sometimes referred to as vascular permeability factor.

In one embodiment, all of the members of the VEGF family stimulate cellular responses by binding to tyrosine kinase receptors (the VEGFRs) on the cell surface, causing them to dimerize and become activated through transphosphorylation. The VEGF receptors have an extracellular portion consisting of 7 immunoglobulin-like domains, a single transmembrane spanning region and an intracellular portion containing a split tyrosine-kinase domain.

In one embodiment, VEGF-A is a VEGFR-2 (KDR/Flk-1) ligand as well as a VEGFR-1 (Flt-1) ligand. In one embodiment, VEGFR-mediates almost all of the known cellular responses to VEGF. The function of VEGFR-1 is less well defined, although it is thought to modulate VEGFR-2 signaling, in one embodiment, via sequestration of VEGF from VEGFR-2 binding, which in one embodiment, is particularly important during vasculogenesis in the embryo. In one embodiment, VEGF-C and VEGF-D are ligands of the VEGFR-3 receptor, which in one embodiment, mediates lymphangiogenesis.

In one embodiment, the compositions of the present disclosure comprise a VEGF receptor or a fragment thereof, which in one embodiment, is a VEGFR-2 and, in another embodiment, a VEGFR-1, and, in another embodiment, VEGFR-3.

In one embodiment, vascular Endothelial Growth Factor Receptor 2 (VEGFR2) is highly expressed on activated endothelial cells (ECs) and participates in the formation of new blood vessels. In one embodiment, VEGFR2 binds all 5 isoforms of VEGF. In one embodiment, signaling of VEGF through VEGFR2 on ECs induces proliferation, migration, and eventual differentiation. In one embodiment, the mouse homologue of VEGFR2 is the fetal liver kinase gene-1 (Flk-1), which is a strong therapeutic target, and has important roles in tumor growth, invasion, and metastasis. In one embodiment, VEGFR2 is also referred to as kinase insert domain receptor (a type III receptor tyrosine kinase) (KDR), cluster of differentiation 309 (CD309), FLK1, Ly73, Krd-1, VEGFR, VEGFR-2, or 6130401C07.

In other embodiments, the antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, the melanoma-associated antigens (TRP-2, MAGE-1, MAGE-3, gp-100, tyrosinase, MART-1, HSP-70, beta-HCG), human papilloma virus antigens E1 and E2 from type HPV-16, -18, -31, -33, -35 or -45 human papilloma viruses, the tumor antigens CEA, the ras protein, mutated or otherwise, the p53 protein, mutated or otherwise, Muc1, or pSA.

In other embodiments, the antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough3 yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and listeriosis.

In another embodiment, an HPV E6 antigen is utilized instead of or in addition to an E7 antigen in a method of the present disclosure for treating, protecting against, or inducing an immune response against a cervical cancer.

In another embodiment, an ActA protein fragment is utilized instead of or in addition to an LLO fragment in a method of the present disclosure for treating, protecting against, or inducing an immune response against a cervical cancer.

In another embodiment, a PEST amino acid sequence-containing protein fragment is utilized instead of or in addition to an LLO fragment in a method of the present disclosure for treating, protecting against, or inducing an immune response against a cervical cancer.

In another embodiment, the present disclosure provides an immunogenic composition comprising a recombinant Listeria of the present disclosure. In another embodiment, the immunogenic composition of methods and compositions of the present disclosure comprises a recombinant vaccine vector of the present disclosure. In another embodiment, the immunogenic composition comprises a plasmid of the present disclosure. In another embodiment, the immunogenic composition comprises an adjuvant. In one embodiment, a vector of the present disclosure may be administered as part of a vaccine composition.

In another embodiment, a vaccine of the present disclosure is delivered with an adjuvant. In one embodiment, the adjuvant favors a predominantly Th1-mediated immune response. In another embodiment, the adjuvant favors a Th1-type immune response. In another embodiment, the adjuvant favors a Th1-mediated immune response. In another embodiment, the adjuvant also stimulates a STING pathway. In another embodiment, the adjuvant favors a cell-mediated immune response over an antibody-mediated response. In another embodiment, the adjuvant is any other type of adjuvant known in the art. In another embodiment, the immunogenic composition induces the formation of a T cell immune response against the target protein.

In another embodiment, an ActA protein fragment is utilized instead of or in addition to an LLO fragment in a method of the present disclosure for treating or ameliorating an HPV-mediated disease, disorder, or symptom.

In another embodiment, a PEST amino acid sequence-containing protein fragment is utilized instead of or in addition to an LLO fragment in a method of the present disclosure for treating or ameliorating an HPV-mediated disease, disorder, or symptom.

In another embodiment, an HPV E6 antigen is utilized instead of or in addition to an E7 antigen in a method of the present disclosure for treating or ameliorating an HPV-mediated disease, disorder, or symptom.

The antigen of methods and compositions of the present disclosure is, in another embodiment, an HPV E7 protein. In another embodiment, the antigen is an HPV E6 protein. In another embodiment, the antigen is any other HPV protein known in the art.

“E7 antigen” refers, in another embodiment, to an E7 protein. In another embodiment, the term refers to an E7 fragment. In another embodiment, the term refers to an E7 peptide. In another embodiment, the term refers to any other type of E7 antigen known in the art.

The E7 protein of methods and compositions of the present disclosure is, in another embodiment, an HPV 16 E7 protein. In another embodiment, the E7 protein is an HPV-18 E7 protein. In another embodiment, the E7 protein is an HPV-31 E7 protein. In another embodiment, the E7 protein is an HPV-35 E7 protein. In another embodiment, the E7 protein is an HPV-39 E7 protein. In another embodiment, the E7 protein is an HPV-45 E7 protein. In another embodiment, the E7 protein is an HPV-51 E7 protein. In another embodiment, the E7 protein is an HPV-52 E7 protein. In another embodiment, the E7 protein is an HPV-58 E7 protein. In another embodiment, the E7 protein is an E7 protein of a high-risk HPV type. In another embodiment, the E7 protein is an E7 protein of a mucosal HPV type.

“E6 antigen” refers, in another embodiment, to an E6 protein. In another embodiment, the term refers to an E6 fragment. In another embodiment, the term refers to an E6 peptide. In another embodiment, the term refers to any other type of E6 antigen known in the art.

The E6 protein of methods and compositions of the present disclosure is, in another embodiment, an HPV 16 E6 protein. In another embodiment, the E6 protein is an HPV-18 E6 protein. In another embodiment, the E6 protein is an HPV-31 E6 protein. In another embodiment, the E6 protein is an HPV-35 E6 protein. In another embodiment, the E6 protein is an HPV-39 E6 protein. In another embodiment, the E6 protein is an HPV-45 E6 protein. In another embodiment, the E6 protein is an HPV-51 E6 protein. In another embodiment, the E6 protein is an HPV-52 E6 protein. In another embodiment, the E6 protein is an HPV-58 E6 protein. In another embodiment, the E6 protein is an E6 protein of a high-risk HPV type. In another embodiment, the E6 protein is an E6 protein of a mucosal HPV type.

The immune response induced by methods and compositions of the present disclosure is, in another embodiment, a T cell response. In another embodiment, the immune response comprises a T cell response. In another embodiment, the response is a CD8⁺ T cell response. In another embodiment, the response comprises a CD8⁺ T cell response. In another embodiment, the response is a CD4⁺ T cell response. In another embodiment, the response comprises a combination of a CD8+ T cell and CD4+ T cell response.

The N-terminal LLO protein fragment of methods and compositions of the present disclosure comprises, in another embodiment, SEQ ID No: 2. In another embodiment, the fragment comprises an LLO signal peptide. In another embodiment, the fragment comprises SEQ ID No: 2. In another embodiment, the fragment consists approximately of SEQ ID No: 2. In another embodiment, the fragment consists essentially of SEQ ID No: 2. In another embodiment, the fragment corresponds to SEQ ID No: 2. In another embodiment, the fragment is homologous to SEQ ID No: 2. In another embodiment, the fragment is homologous to a fragment of SEQ ID No: 2. The ΔLLO used in some of the Examples was 416 AA long (exclusive of the signal sequence), as 88 residues from the amino terminus which is inclusive of the activation domain containing cysteine 484 were truncated. It will be clear to those skilled in the art that any ΔLLO without the activation domain, and in particular without cysteine 484, are suitable for methods and compositions of the present disclosure. In another embodiment, fusion of an E7 or E6 antigen to any ΔLLO, including the PEST amino acid AA sequence, SEQ ID NO: 18, enhances cell mediated and anti-tumor immunity of the antigen.

The LLO protein utilized to construct vaccines of the present disclosure has, in another embodiment, the sequence:

MKKIMLVFITLILVSLPIAQQTEAKDASAFNKENSISSMAPPASPPASPKTPIEKKH ADEIDKYIQGLDYNKNNVLVYHGDAVTNVPPRKGYKDGNEYIVVEKKKKSINQ NNADIQVVNAISSLTYPGALVKANSELVENQPDVLPVKRDSLTLSIDLPGMTNQD NKIVVKNATKSNVNNAVNTLVERWNEKYAQAYPNVSAKIDYDDEMAYSESQLI AKFGTAFKAVNNSLNVNFGAISEGKMQEEVISFKQIYYNVNVNEPTRPSRFFGKA VTKEQLQALGVNAENPPAYISSVAYGRQVYLKLSTNSHSTKVKAAFDAAVSGKS VSGDVELTNIIKNSSFKAVIYGGSAKDEVQIIDGNLGDLRDILKKGATFNRETPGV PIAYTTNFLKDNELAVIKNNSEYIETTSKAYTDGKINIDHSGGYVAQFNISWDEVN YDPEGNEIVQHKNWSENNKSKLAHFTSSIYLPGNARNINVYAKECTGLAWEWW RTVIDDRNLPLVKNRNISIWGTTLYPKYSNKVDNPIE (GenBank Accession No. P13128; SEQ ID NO: 1; nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a vaccine of the present disclosure.

In another embodiment, the N-terminal fragment of an LLO protein utilized in compositions and methods of the present disclosure has the sequence:

(SEQ ID NO: 2)
MKKIMLVFITLILVSLPIAQQTEAKDASAFNKENSISSVAPPASPPASP
KTPIEKKHADEIDKYIQGLDYNKNNVLVYHGDAVTNVPPRKGYKDGNEY
IVVEKKKKSINQNNADIQVVNAISSLTYPGALVKANSELVENQPDVLPV
KRDSLTLSIDLPGMTNQDNKIVVKNATKSNVNNAVNTLVERWNEKYAQA
YSNVSAKIDYDDEMAYSESQLIAKFGTAFKAVNNSLNVNFGAISEGKMQ
EEVISFKQIYYNVNVNEPTRPSRFFGKAVTKEQLQALGVNAENPPAYIS
SVAYGRQVYLKLSTNSHSTKVKAAFDAAVSGKSVSGDVELTNIIKNSSF
KAVIYGGSAKDEVQIIDGNLGDLRDILKKGATFNRETPGVPIAYTTNFL
KDNELAVIKNNSEYIETTSKAYTDGKINIDHSGGYVAQFNISWDEVNYD.

In another embodiment, the LLO fragment corresponds to about AA 20-442 of an LLO protein utilized herein.

In another embodiment, the LLO fragment has the sequence:

(SEQ ID NO: 3)
MKKIMLVFITLILVSLPIAQQTEAKDASAFNKENSISSVAPPASPPASP
KTPIEKKHADEIDKYIQGLDYNKNNVLVYHGDAVTNVPPRKGYKDGNEY
IVVEKKKKSINQNNADIQVVNAISSLTYPGALVKANSELVENQPDVLPV
KRDSLTLSIDLPGMTNQDNKIVVKNATKSNVNNAVNTLVERWNEKYAQA
YSNVSAKIDYDDEMAYSESQLIAKFGTAFKAVNNSLNVNFGAISEGKMQ
EEVISFKQIYYNVNVNEPTRPSRFFGKAVTKEQLQALGVNAENPPAYIS
SVAYGRQVYLKLSTNSHSTKVKAAFDAAVSGKSVSGDVELTNIIKNSSF
KAVIYGGSAKDEVQIIDGNLGDLRDILKKGATFNRETPGVPIAYTTNFL
KDNELAVIKNNSEYIETTSKAYTD.

In another embodiment, “truncated LLO” or “ΔLLO” refers to a fragment of LLO that comprises the PEST amino acid domain. In another embodiment, the terms refer to an LLO fragment that comprises a PEST sequence.

In another embodiment, the terms refer to an LLO fragment that does not contain the activation domain at the amino terminus and does not include cysteine 484. In another embodiment, the terms refer to an LLO fragment that is not hemolytic. In another embodiment, the LLO fragment is rendered non-hemolytic by deletion or mutation of the activation domain. In another embodiment, the LLO fragment is rendered non-hemolytic by deletion or mutation of cysteine 484. In another embodiment, the LLO fragment is rendered non-hemolytic by deletion or mutation at another location.

In another embodiment, the LLO fragment consists of about the first 441 AA of the LLO protein. In another embodiment, the LLO fragment consists of about the first 420 AA of LLO. In another embodiment, the LLO fragment is a non-hemolytic form of the LLO protein.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein, then the residue numbers can be adjusted accordingly.

In another embodiment, the LLO fragment is any other LLO fragment known in the art.

In another embodiment, an ActA protein comprises SEQ ID NO: 4 MGLNRFMRAMMVVFITANCITINPDIIFAATDSE DSSLNTDEWEEEKTEEQPSEVNTGPRYETARE SSRDIEELEKSNKVKNTNKADLIAMLKAKAE KGPNNNNNNGEQTGNVAINEEASGVDRPTLQV ERRHPGLSSDSAAEIKKRRKAIASSDSELESLT YPDKPTKANKRKVAKESVVDASESDLDSSMQS ADESTPQPLKANQKPFFPKVFKKIKDAGKWVR DKIDENPEVKKAIVDKS AGLIDQLLTKKKSEEV NASDFPPPPTDEELRLALPETPMLLGFNAPTPS EPSSFEFPPPPTDEELRLALPETPMLLGFNAPA TSEPSSFEFPPPPTEDELEIMRETAPSLDSSFTS GDLASLRSAINRHSENFSDFPPIPTEEELNGRG GRPTSEEFSSLNSGDFTDDENSETTEEEIDRLA DLRDRGTGKHSRNAGFLPLNPFISSPVPSLTPK VPKISAPALISDITKKAPFKNPSQPLNVFNKKT TTKTVTKKPTPVKTAPKLAELPATKPQETVLR ENKTPFIEKQAETNKQSINMPSLPVIQKEATES DKEEMKPQTEEKMVEESES ANNANGKNRSAGI EEGKLIAKSAEDEKAKEEPGNHTTLILAMLAIG VFSLGAFIKIIQLRKNN (SEQ ID NO: 4). The first 29 AA of the proprotein corresponding to this sequence are the signal sequence and are cleaved from ActA protein when it is secreted by the bacterium. In one embodiment, an ActA polypeptide or peptide comprises the signal sequence, AA 1-29 of SEQ ID NO: 4. In another embodiment, an ActA polypeptide or peptide does not include the signal sequence, AA 1-29 of SEQ ID NO: 4.

In one embodiment, an ActA protein comprises SEQ ID NO: 5

MGLNRFMRAMMVVFITANCITINPDIIFAATDSEDSSLNTDEWEEEKTEEQ PSEVNTGPRYETAREVSSRDIKELEKSNKVRNTNKADLIAMLKEKAEKGPNINNN NSEQTENAAINEEASGADRPAIQVERRHPGLPSDSAAEIKKRRKAIASSDSELESLT YPDKPTKVNKKKVAKESVADASESDLDSSMQSADESSPQPLKANQQPFFPKVFK KIKDAGKWVRDKIDENPEVKKAIVDKSAGLIDQLLTKKKSEEVNASDFPPPPTDE ELRLALPETPMLLGFNAPATSEPSSFEFPPPPTDEELRLALPETPMLLGFNAPATSE PSSFEFPPPPTEDELEIIRETASSLDSSFTRGDLASLRNAINRHSQNFSDFPPIPTEEEL NGRGGRPTSEEFSSLNSGDFTDDENSETTEEEIDRLADLRDRGTGKHSRNAGFLPL NPFASSPVPSLSPKVSKISDRALISDITKKTPFKNPSQPLNVFNKKTTTKTVTKKPTP VKTAPKLAELPATKPQETVLRENKTPFIEKQAETNKQSINMPSLPVIQKEATESDK EEMKPQTEEKMVEESESANNANGKNRSAGIEEGKLIAKSAEDEKAKEEPGNHTT LILAMLAIGVFSLGAFIKIIQLRKNN (SEQ ID NO: 5). In another embodiment, an ActA protein comprises SEQ ID NO: 5. The first 29 AA of the proprotein corresponding to this sequence are the signal sequence and are cleaved from ActA protein when it is secreted by the bacterium. In one embodiment, an ActA polypeptide or peptide comprises the signal sequence, AA 1-29 of SEQ ID NO: 5 above. In another embodiment, an ActA polypeptide or peptide does not include the signal sequence, AA 1-29 of SEQ ID NO: 5 above.

In one embodiment, a truncated ActA protein comprises an N-terminal fragment of an ActA protein. In another embodiment, a truncated ActA protein is an N-terminal fragment of an ActA protein. In one embodiment, a truncated ActA protein comprises SEQ ID NO: 6

(SEQ ID NO: 6)
MRAMMVVFITANCITINPDIIFAATDSEDSSLNTDEWEEEKTEEQPSEV
NTGPRYETAREVSSRDIKELEKSNKVRNTNKADLIAMLKEKAEKGPNIN
NNNSEQTENAMNEEASGADRPAIQVERRHPGLPSDSAAEIKKRRKAIAS
SDSELESLTYPDKPTKVNKKKVAKESVADASESDLDSSMQSADESSPQP
LKANQQPFFPKVFKKIKDAGKWVRDKIDENPEVKKAIVDKSAGLIDQLL
TKKKSEEVNASDFPPPPTDEELRLALPETPMLLGFNAPATSEPSSFEFP
PPPTDEELRLALPETPMLLGFNAPATSEPSSFEFPPPPTEDELEIIRET
ASSLDSSFTRGDLASLRNAINRHSQNFSDFPPIPTEEELNGRGGRP.

In another embodiment, the ActA fragment comprises the sequence set forth in SEQ ID NO: 6.

In another embodiment, a truncated ActA protein comprises SEQ ID NO: 7:

(SEQ ID NO: 7)
MGLNRFMRAMMVVFITANCITINPDIIFAATDSEDSSLNTDEWEEEKTE
EQPSEVNTGPRYETAREVSSRDIKELEKSNKVRNTNKADLIAMLKEKAE
KG.

In another embodiment, the ActA fragment is any other ActA fragment known in the art.

In one embodiment, a truncated ActA protein comprises SEQ ID NO: 8

(SEQ ID NO: 8)
MRAMMVVFITANCITINPDIIFAATDSEDSSLNTDEWEEEKTEEQPSEV
NTGPRYETAREVSSRDIKELEKSNKVRNTNKADLIAMLKEKAEKGPNIN
NNNSEQTENAAINEEASGADRPAIQVERRHPGLPSDSAAEIKKRRKAIA
SSDSELESLTYPDKPTKVNKKKVAKESVADASESDLDSSMQSADESSPQ
PLKANQQPFFPKVFKKIKDAGKWVRDKIDENPEVKKAIVDKSAGLIDQL
LTKKKSEEVNASDFPPPPTDEELRLALPETPMLLGFNAPATSEPSSFEF
PPPPTDEELRLALPETPMLLGFNAPATSEPSSFEFPPPPTEDELEIIRE
TASSLDSSFTRGDLASLRNAINRHSQNFSDFPPIPTEEELNGRGGRP.

In another embodiment, a truncated ActA protein comprises SEQ ID NO: 9: MGLNRFMRAMMVVFITANCITINPDITAATDSEDSSLNTDEWEEEKTEEQPSE VNTGPRYETAREVSSRDIKELEKSNKVRNTNKADLIAMLKEKAEKG (SEQ ID NO: 9).

In another embodiment, a truncated ActA protein comprises SEQ ID NO: 10 ATDSEDSSLNTDEWEEEKTEEQPSEVNTGPRY ETAREVSSRDIEELEKSNKVKNTNKADLIAML KAKAEKGPNNNNNNGEQTGNVAINEEASG (SEQ ID NO: 10). In another embodiment, a truncated ActA as set forth in SEQ ID NO: 10 is referred to as ActA/PEST1. In another embodiment, a truncated ActA comprises from the first 30 to amino acid 122 of the full length ActA sequence. In another embodiment, SEQ ID NO: 10 comprises from the first 30 to amino acid 122 of the full length ActA sequence. In another embodiment, a truncated ActA comprises from the first 30 to amino acid 122 of SEQ ID NO: 10. In another embodiment, SEQ ID NO: 10 comprises from the first 30 to amino acid 122 of SEQ ID NO: 4.

In another embodiment, a truncated ActA protein comprises SEQ ID NO: 11 ATDSEDSSLNTDEWEEEKTEEQPSEVNTGPRY ETAREVSSRDIEELEKSNKVKNTNKADLIAML KAKAEKGPNNNNNNGEQTGNVAINEEASGVD RPTLQVERRHPGLSSDSAAEIKKRRKAIASSDS ELESLTYPDKPTKANKRKVAKESVVDASESDL DSSMQSADESTPQPLKANQKPFFPKVFKKIKD AGKWVRDK (SEQ ID NO: 11). In another embodiment, a truncated ActA as set forth in SEQ ID NO: 11 is referred to as ActA/PEST2. In another embodiment, a truncated ActA comprises from amino acid 30 to amino acid 229 of the full length ActA sequence. In another embodiment, SEQ ID NO: 11 comprises from about amino acid 30 to about amino acid 229 of the full length ActA sequence. In another embodiment, a truncated ActA comprises from about amino acid 30 to amino acid 229 of SEQ ID NO: 4. In another embodiment, SEQ ID NO: 11 comprises from amino acid 30 to amino acid 229 of SEQ ID NO: 4.

In another embodiment, a truncated ActA protein comprises SEQ ID NO: 12 ATDSEDSSLNTDEWEEEKTEEQPSEVNTGPRY ETAREVSSRDIEELEKSNKVKNTNKADLIAML KAKAEKGPNNNNNNGEQTGNVAINEEASGVD RPTLQVERRHPGLSSDSAAEIKKRRKAIASSDS ELESLTYPDKPTKANKRKVAKESVVDASESDL DSSMQSADESTPQPLKANQKPFFPKVFKKIKD AGKWVRDKIDENPEVKKAIVDKSAGLIDQLLT KKKSEEVNASDFPPPPTDEELRLALPETPMLLG FNAPTPSEPSSFEFPPPPTDEELRLALPETPMLL GFNAPATSEPSS (SEQ ID NO: 12). In another embodiment, a truncated ActA as set forth in SEQ ID NO: 12 is referred to as ActA/PEST3. In another embodiment, this truncated ActA comprises from the first 30 to amino acid 332 of the full length ActA sequence. In another embodiment, SEQ ID NO: 12 comprises from the first 30 to amino acid 332 of the full length ActA sequence. In another embodiment, a truncated ActA comprises from about the first 30 to amino acid 332 of SEQ ID NO: 4. In another embodiment, SEQ ID NO: 12 comprises from the first 30 to amino acid 332 of SEQ ID NO: 4.

In another embodiment, a truncated ActA protein comprises SEQ ID NO: 13 ATDSEDSSLNTDEWEEEKTEEQPSEVNTGPRY ETAREVSSRDIEELEKSNKVKNTNKADLIAML KAKAEKGPNNNNNNGEQTGNVAINEEASGVD RPTLQVERRHPGLSSDSAAEIKKRRKAIASSDS ELESLTYPDKPTKANKRKVAKESVVDASESDL DSSMQSADESTPQPLKANQKPFFPKVFKKIKD AGKWVRDKIDENPEVKKAIVDKSAGLIDQLLT KKKSEEVNASDFPPPPTDEELRLALPETPMLLG FNAPTPSEPSSFEFPPPPTDEELRLALPETPMLL GFNAPATSEPSSFEFPPPPTEDELEIMRETAPSL DSSFTSGDLASLRSAINRHSENFSDFPLIPTEEE LNGRGGRPTSE (SEQ ID NO: 13). In another embodiment, a truncated ActA as set forth in SEQ ID NO: 13 is referred to as ActA/PEST4. In another embodiment, this truncated ActA comprises from the first 30 to amino acid 399 of the full length ActA sequence. In another embodiment, SEQ ID NO: 13 comprises from the first 30 to amino acid 399 of the full length ActA sequence. In another embodiment, a truncated ActA comprises from the first 30 to amino acid 399 of SEQ ID NO: 4. In another embodiment, SEQ ID NO: 13 comprises from the first 30 to amino acid 399 of SEQ ID NO: 4.

In another embodiment, a truncated ActA sequence disclosed herein is further fused to an hly signal peptide at the N-terminus. In another embodiment, the truncated ActA fused to hly signal peptide comprises SEQ ID NO: 14

MKKIMLVFITLILVSLPIAQQTEASRATDS EDSSLNTDEWEEEKTEEQPSEVNTGPRYETAR EVSSRDIEELEKSNKVKNTNKADLIAMLKAKA EKGPNNNNNNGEQTGNVAINEEASGVDRPTLQ ERRHPGLSSDSAAEIKKRRKAIASSDSELESL TYPDKPTKANKRKVAKESVVDASESDLDSSMQ SADESTPQPLKANQKPFFPKVFKKIKDAGKWV RDK. In another embodiment, a truncated ActA as set forth in SEQ ID NO: 14 is referred to as LA229.

In another embodiment, a recombinant nucleotide encoding a truncated ActA protein disclosed herein comprises SEQ ID NO: 15 Atgcgtgcgatgatggtggttttcattactgccaattgcattacgattaaccccgacataatatttgcagcgacagatagcgaagattct agtctaaacacagatgaatgggaagaagaaaaaacagaagagcaaccaagcgaggtaaatacgggaccaagatacgaaactgca cgtgaagtaagttcacgtgatattaaagaactagaaaaatcgaataaagtgagaaatacgaacaaagcagacctaatagcaatgttga aagaaaaagcagaaaaaggtccaaatatcaataataacaacagtgaacaaactgagaatgcggctataaatgaagaggcttcagga gccgaccgaccagctatacaagtggagcgtcgtcatccaggattgccatcggatagcgcagcggaaattaaaaaaagaaggaaag ccatagcatcatcggatagtgagcttgaaagccttacttatccggataaaccaacaaaagtaaataagaaaaaagtggcgaaagagt cagttgcggatgcttctgaaagtgacttagattctagcatgcagtcagcagatgagtcttcaccacaacctttaaaagcaaaccaacaa ccatttttccctaaagtatttaaaaaaataaaagatgcggggaaatgggtacgtgataaaatcgacgaaaatcctgaagtaaagaaagc gattgttgataaaagtgcagggttaattgaccaattattaaccaaaaagaaaagtgaagaggtaaatgcttcggacttcccgccaccac ctacggatgaagagttaagacttgctttgccagagacaccaatgcttcttggttttaatgctcctgctacatcagaaccgagctcattcga atttccaccaccacctacggatgaagagttaagacttgctttgccagagacgccaatgcttcttggttttaatgctcctgctacatcggaa ccgagctcgttcgaatttccaccgcctccaacagaagatgaactagaaatcatccgggaaacagcatcctcgctagattctagttttac aagaggggatttagctagtttgagaaatgctattaatcgccatagtcaaaatttctctgatttcccaccaatcccaacagaagaagagtt gaacgggagaggcggtagacca (SEQ ID NO: 15). In another embodiment, the recombinant nucleotide has the sequence set forth in SEQ ID NO: 15. In another embodiment, the recombinant nucleotide comprises any other sequence that encodes a fragment of an ActA protein.

In another embodiment, a truncated ActA fused to hly signal peptide is encoded by a sequence comprising SEQ ID NO: 16

Atgaaaaaaataatgctagtttttattacacttatattagttagtctaccaattgcgcaacaaactgaagcatctagagcgac agatagcgaagattccagtctaaacacagatgaatgggaagaagaaaaaacagaagagcagccaagcgaggtaaatacgggac caagatacgaaactgcacgtgaagtaagttcacgtgatattgaggaactagaaaaatcgaataaagtgaaaaatacgaacaaagca gacctaatagcaatgttgaaagcaaaagcagagaaaggtccgaataacaataataacaacggtgagcaaacaggaaatgtggctat aaatgaagaggcttcaggagtcgaccgaccaactctgcaagtggagcgtcgtcatccaggtctgtcatcggatagcgcagcggaa attaaaaaaagaagaaaagccatagcgtcgtcggatagtgagcttgaaagccttacttatccagataaaccaacaaaagcaaataag agaaaagtggcgaaagagtcagttgtggatgcttctgaaagtgacttagattctagcatgcagtcagcagacgagtctacaccacaa cctttaaaagcaaatcaaaaaccatttttccctaaagtatttaaaaaaataaaagatgcggggaaatgggtacgtgataaa (SEQ ID NO: 16). In another embodiment, SEQ ID NO: 16 comprises a sequence encoding a linker region (see bold, italic text) that is used to create a unique restriction enzyme site for XbaI so that different polypeptides, heterologous antigens, etc. can be cloned after the signal sequence. Hence, it will be appreciated by a skilled artisan that signal peptidases act on the sequences before the linker region to cleave signal peptide.

In another embodiment, the recombinant nucleotide encoding a truncated ActA protein comprises the sequence set forth in SEQ ID NO: 17

(SEQ ID NO: 17)
atgcgtgcgatgatggtggttttcattactgccaattgcattacgatta
accccgacataatatttgcagcgacagatagcgaagattctagtctaaa
cacagatgaatgggaagaagaaaaaacagaagagcaaccaagcgaggta
aatacgggaccaagatacgaaactgcacgtgaagtaagttcacgtgata
ttaaagaactagaaaaatcgaataaagtgagaaatacgaacaaagcaga
cctaatagcaatgttgaaagaaaaagcagaaaaaggtccaaatatcaat
aataacaacagtgaacaaactgagaatgcggctataaatgaagaggctt
caggagccgaccgaccagctatacaagtggagcgtcgtcatccaggatt
gccatcggatagcgcagcggaaattaaaaaaagaaggaaagccatagca
tcatcggatagtgagcttgaaagccttacttatccggataaaccaacaa
aagtaaataagaaaaaagtggcgaaagagtcagttgcggatgcttctga
aagtgacttagattctagcatgcagtcagcagatgagtcttcaccacaa
cctttaaaagcaaaccaacaaccatttttccctaaagtatttaaaaaaa
taaaagatgcggggaaatgggtacgtgataaaatcgacgaaaatcctga
agtaaagaaagcgattgttgataaaagtgcagggttaattgaccaatta
ttaaccaaaaagaaaagtgaagaggtaaatgcttcggacttcccgccac
cacctacggatgaagagttaagacttgctttgccagagacaccaatgct
tcttggttttaatgctcctgctacatcagaaccgagctcattcgaattt
ccaccaccacctacggatgaagagttaagacttgctttgccagagacgc
caatgcttcttggttttaatgctcctgctacatcggaaccgagctcgtt
cgaatttccaccgcctccaacagaagatgaactagaaatcatccgggaa
acagcatcctcgctagattctagttttacaagaggggatttagctagtt
tgagaaatgctattaatcgccatagtcaaaatttctctgatttcccacc
aatcccaacagaagaagagttgaacgggagaggcggtagacca.

In another embodiment, the recombinant nucleotide has the sequence set forth in SEQ ID NO: 17. In another embodiment, the recombinant nucleotide comprises other sequences that encode a fragment of an ActA protein.

In another embodiment, a truncated ActA protein is a fragment of an ActA protein. In another embodiment, the truncated ActA protein is an N-terminal fragment of an ActA protein. In another embodiment, the terms “truncated ActA,” “N-terminal ActA fragment” or “ΔActA” are used interchangeably herein and refer to a fragment of ActA that comprises a PEST domain. In another embodiment, the terms refer to an ActA fragment that comprises a PEST sequence. In another embodiment, the terms refer to an immunogenic fragment of the ActA protein. In another embodiment, the terms refer to a truncated ActA fragment encoded by SEQ ID NO: 5-14 disclosed herein.

The N-terminal ActA protein fragment of methods and compositions of the present disclosure comprises, in one embodiment, a sequence selected from SEQ ID No: 5-14. In another embodiment, the ActA fragment comprises an ActA signal peptide. In another embodiment, the ActA fragment consists approximately of a sequence selected from SEQ ID NO: 56-14. In another embodiment, the ActA fragment consists essentially of a sequence selected from SEQ ID NO: 5-14. In another embodiment, the ActA fragment corresponds to a sequence selected from SEQ ID NO: 5-14. In another embodiment, the ActA fragment is homologous to a sequence selected from SEQ ID NO: 5-14.

In another embodiment, a PEST-sequence is any PEST-AA sequence derived from a prokaryotic organism. The PEST-sequence may be other PEST-sequences known in the art.

In another embodiment, an ActA fragment consists of about the first 100 AA of the wild-type ActA protein. In another embodiment, an ActA fragment consists of about the first 100 AA of an ActA protein disclosed herein.

In another embodiment, the ActA fragment consists of about residues 1-25, 1-50, 1-75, 1-100, 1-125, 1-150, 1-175, 1-200, 1-225, 1-250, 1-275, 1-300, 1-325, 1-338, 1-350, 1-375, 1-400, 1-450, 1-500, 1-550, 1-600, 1-639. In another embodiment, the ActA fragment consists of about residues 30-100, 30-125, 30-150, 30-175, 30-200, 30-225, 30-250, 30-275, 30-300, 30-325, 30-338, 30-350, 30-375, 30-400, 30-450, 30-500, 30-550, 30-600, or 30-604.

In another embodiment, an ActA fragment disclosed herein contains residues of a homologous ActA protein that correspond to one of the above AA ranges. The residue numbers need not, in another embodiment, correspond exactly with the residue numbers enumerated above; e.g. if the homologous ActA protein has an insertion or deletion, relative to an ActA protein utilized herein, then the residue numbers can be adjusted accordingly.

In another embodiment, a homologous ActA refers to identity of an ActA sequence (e.g. to one of SEQ ID NO: 4-17) of greater than 70%. In another embodiment, a homologous ActA refers to identity to one of SEQ ID NO: 4-17 of greater than 72%. In another embodiment, a homologous refers to identity to one of SEQ ID No: 4-17 of greater than 75%. In another embodiment, a homologous refers to identity to one of SEQ ID NO: 4-17 of greater than 78%. In another embodiment, a homologous refers to identity to one of SEQ ID NO: 4-17 of greater than 80%. In another embodiment, a homologous refers to identity to one of SEQ ID NO: 4-17 of greater than 82%. In another embodiment, a homologous refers to identity to one of SEQ ID NO: 4-17 of greater than 83%. In another embodiment, a homologous refers to identity to one of SEQ ID NO: 4-17 of greater than 85%. In another embodiment, a homologous refers to identity to one of SEQ ID NO: 4-17 of greater than 87%. In another embodiment, a homologous refers to identity to one of SEQ ID NO: 4-17 of greater than 88%. In another embodiment, a homologous refers to identity to one of SEQ ID NO: 4-17 of greater than 90%. In another embodiment, a homologous refers to identity to one of SEQ ID NO: 4-17 of greater than 92%. In another embodiment, a homologous refers to identity to one of SEQ ID NO: 4-17 of greater than 93%. In another embodiment, a homologous refers to identity to one of SEQ ID NO: 4-17 of greater than 95%. In another embodiment, a homologous refers to identity to one of SEQ ID NO: 4-17 of greater than 96%. In another embodiment, a homologous refers to identity to one of SEQ ID NO: 4-17 of greater than 97%. In another embodiment, a homologous refers to identity to one of SEQ ID NO: 4-17 of greater than 98%. In another embodiment, a homologous refers to identity to one of SEQ ID NO: 4-17 of greater than 99%. In another embodiment, a homologous refers to identity to one of SEQ ID NO: 4-17 of 100%.

In another embodiment of the methods and compositions of the present disclosure, a PEST amino acid AA sequence is fused to the E7 or E6 antigen. As provided herein, recombinant Listeria strains expressing PEST amino acid sequence-antigen fusions induce anti-tumor immunity (Example 3) and generate antigen-specific, tumor-infiltrating T cells (Example 4). Further, enhanced cell mediated immunity was demonstrated for fusion proteins comprising an antigen and LLO containing the PEST amino acid AA sequence KENSISSMAPPASPPASPKTPIEKKHADEIDK (SEQ ID NO: 18).

Thus, fusion of an antigen to other LM PEST amino acid sequences and PEST amino acid sequences derived from other prokaryotic organisms will also enhance immunogenicity of the antigen. The PEST amino acid AA sequence has, in another embodiment, a sequence selected from SEQ ID NO: 19-24. In another embodiment, the PEST amino acid sequence is a PEST amino acid sequence from the LM ActA protein. In another embodiment, the PEST amino acid sequence is KTEEQPSEVNTGPR (SEQ ID NO: 19), KASVTDTSEGDLDSSMQSADESTPQPLK (SEQ ID NO: 20), KNEEVNASDFPPPPTDEELR (SEQ ID NO: 21), or RGGIPTSEEFSSLNSGDFTDDENSETTEEEIDR (SEQ ID NO: 22). In another embodiment, the PEST amino acid sequence is from Streptolysin O protein of Streptococcus sp. In another embodiment, the PEST amino acid sequence is from Streptococcus pyogenes Streptolysin O, e.g. KQNTASTETTTTNEQPK (SEQ ID NO: 23) at AA 35-51. In another embodiment, the PEST amino acid sequence is from Streptococcus equisimilis Streptolysin O, e.g. KQNTANTETTTTNEQPK (SEQ ID NO: 24) at AA 38-54. In another embodiment, the PEST amino acid sequence is another PEST amino acid AA sequence derived from a prokaryotic organism. In another embodiment, the PEST amino acid sequence is any other PEST amino acid sequence known in the art.

PEST amino acid sequences of other prokaryotic organism can be identified in accordance with methods such as described by, for example Rechsteiner and Rogers (1996, Trends Biochem. Sci. 21:267-271) for LM. Alternatively, PEST amino acid AA sequences from other prokaryotic organisms can also be identified based by this method. Other prokaryotic organisms wherein PEST amino acid AA sequences would be expected to include, but are not limited to, other Listeria species. In another embodiment, the PEST amino acid sequence is embedded within the antigenic protein. Thus, in another embodiment, “fusion” refers to an antigenic protein comprising both the antigen and the PEST amino acid amino acid sequence either linked at one end of the antigen or embedded within the antigen.

In another embodiment, the PEST amino acid sequence is identified using any other method or algorithm known in the art, e.g the CaSPredictor (Garay-Malpartida H M, Occhiucci J M, Alves J, Belizario J E. Bioinformatics. 2005 June; 21 Suppl 1:i169-76). In another embodiment, the following method is used:

A PEST index is calculated for each 30-35 AA stretch by assigning a value of 1 to the amino acids Ser, Thr, Pro, Glu, Asp, Asn, or Gln. The coefficient value (CV) for each of the PEST residue is 1 and for each of the other AA (non-PEST) is 0.

In another embodiment, the LLO protein, ActA protein, or fragment thereof of the present disclosure need not be that which is set forth exactly in the sequences set forth herein, but rather other alterations, modifications, or changes can be made that retain the functional characteristics of an LLO or ActA protein fused to an antigen as set forth elsewhere herein. In another embodiment, the present disclosure utilizes an analog of an LLO protein, ActA protein, or fragment thereof. Analogs differ, in another embodiment, from naturally occurring proteins or peptides by conservative AA sequence differences or by modifications which do not affect sequence, or by both.

In one embodiment, the recombinant Listeria strain provided herein is administered to a subject at a dose of 1×10⁹-3.31×10¹⁰ CFU. In another embodiment, the dose is 5-500×10⁸ CFU. In another embodiment, the dose is 7-500×10⁸ CFU. In another embodiment, the dose is 10-500×10⁸ CFU. In another embodiment, the dose is 20-500×10⁸ CFU. In another embodiment, the dose is 30-500×10⁸ CFU. In another embodiment, the dose is 50-500×10⁸ CFU. In another embodiment, the dose is 70-500×10⁸ CFU. In another embodiment, the dose is 100-500×10⁸ CFU. In another embodiment, the dose is 150-500×10⁸ CFU. In another embodiment, the dose is 5-300×10⁸ CFU. In another embodiment, the dose is 5-200×10⁸ CFU. In another embodiment, the dose is 5-150×10⁸ CFU. In another embodiment, the dose is 5-100×10⁸ CFU. In another embodiment, the dose is 5-70×10⁸ CFU. In another embodiment, the dose is 5-50×10⁸ CFU. In another embodiment, the dose is 5-30×10⁸ CFU. In another embodiment, the dose is 5-20×10⁸ CFU. In another embodiment, the dose is 1-30×10⁹ CFU. In another embodiment, the dose is 1-20×10⁹ CFU. In another embodiment, the dose is 2-30×10⁹ CFU. In another embodiment, the dose is 1-10×10⁹ CFU. In another embodiment, the dose is 2-10×10⁹ CFU. In another embodiment, the dose is 3-10×10⁹ CFU. In another embodiment, the dose is 2-7×10⁹ CFU. In another embodiment, the dose is 2-5×10⁹ CFU. In another embodiment, the dose is 3-5×10⁹ CFU.

In another embodiment, the dose is 1×10⁹ organisms. In another embodiment, the dose is 1.5×10⁹ organisms. In another embodiment, the dose is 2×10⁹ organisms. In another embodiment, the dose is 3×10⁹ organisms. In another embodiment, the dose is 4×10⁹ organisms. In another embodiment, the dose is 5×10⁹ organisms. In another embodiment, the dose is 6×10⁹ organisms. In another embodiment, the dose is 7×10⁹ organisms. In another embodiment, the dose is 8×10⁹ organisms. In another embodiment, the dose is 10×10⁹ organisms. In another embodiment, the dose is 1.5×10¹⁰ organisms. In another embodiment, the dose is 2×10¹⁰ organisms. In another embodiment, the dose is 2.5×10¹⁰ organisms. In another embodiment, the dose is 3×10¹⁰ organisms. In another embodiment, the dose is 3.3×10¹⁰ organisms. In another embodiment, the dose is 4×10¹⁰ organisms. In another embodiment, the dose is 5×10¹⁰ organisms.

In another embodiment, a recombinant polypeptide of the methods of the present disclosure is expressed by the recombinant Listeria strain. In another embodiment, the expression is mediated by a nucleotide molecule carried by the recombinant Listeria strain.

In another embodiment, a recombinant Listeria strain provided herein expresses a recombinant polypeptide of the present disclosure by means of a plasmid that encodes the recombinant polypeptide. In another embodiment, the plasmid comprises a gene encoding a bacterial transcription factor. In another embodiment, the plasmid encodes a Listeria transcription factor. In another embodiment, the transcription factor is prfA. In another embodiment, the prfA is a mutant prfA. In another embodiment, the prfA contains a D133V amino acid mutation. In another embodiment, the transcription factor is any other transcription factor known in the art.

In another embodiment, the plasmid comprises a gene encoding a metabolic enzyme. In another embodiment, the metabolic enzyme is a bacterial metabolic enzyme. In another embodiment, the metabolic enzyme is a Listerial metabolic enzyme. In another embodiment, the metabolic enzyme is an amino acid metabolism enzyme. In another embodiment, the amino acid metabolism gene is involved in a cell wall synthesis pathway. In another embodiment, the metabolic enzyme is the product of a D-amino acid aminotransferase gene (dat). In another embodiment, the metabolic enzyme is the product of an alanine racemase gene (dal). In another embodiment, the metabolic enzyme complements a mutation in the genome of said Listeria strain. In another embodiment, the metabolic enzyme is any other metabolic enzyme known in the art. In another embodiment, a method of present disclosure further comprises the step of boosting the subject with a composition comprising a recombinant Listeria strain of the present disclosure. In another embodiment, the recombinant Listeria strain used in the booster inoculation is the same as the strain used in the initial “priming” inoculation. In another embodiment, the booster strain is different from the priming strain. In another embodiment, the same doses are used in the priming and boosting inoculations. In another embodiment, a larger dose is used in the booster. In another embodiment, a smaller dose is used in the booster.

In another embodiment, a method or use of present disclosure further comprises the step of inoculating the subject with an immunogenic composition comprising a heterologous antigen provided herein.

“Boosting” refers, in another embodiment, to administration of an additional vaccine dose to a subject. In another embodiment of methods of the present disclosure, 2 boosts (or a total of at least 3 inoculations) are administered. In another embodiment, 3 boosts are administered. In another embodiment, 4 boosts are administered. In another embodiment, 5 boosts are administered. In another embodiment, 6 boosts are administered. In another embodiment, more than 6 boosts are administered. In another embodiment, the methods of the present disclosure further comprise the step of administering to the subject a booster vaccination. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations. In one embodiment, the period between a prime and a boost vaccine is experimentally determined by the skilled artisan. In another embodiment, the period between a prime and a boost vaccine is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost vaccine is administered 8-10 weeks after the prime vaccine.

The recombinant Listeria strain of methods and compositions of the present disclosure is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art. Each possibility represents a separate embodiment of the present disclosure.

The present disclosure provides a number of listerial species and strains for making or engineering an attenuated Listeria of the present disclosure. In one embodiment, the Listeria strain is L. monocytogenes 10403S wild type (see Bishop and Hinrichs (1987) J. Immunol. 139: 2005-2009; Lauer, et al. (2002) J. Bact. 184: 4177-4186.) In another embodiment, the Listeria strain is L. monocytogenes DP-L4056 (phage cured) (see Lauer, et al. (2002) J. Bact. 184: 4177-4186). In another embodiment, the Listeria strain is L. monocytogenes DP-L4027, which is phage cured and deleted in the hly gene (see Lauer, et al. (2002) J. Bact. 184: 4177-4186; Jones and Portnoy (1994) Infect. Immunity 65: 5608-5613.). In another embodiment, the Listeria strain is L. monocytogenes DP-L4029, which is phage cured, deleted in ActA (see Lauer, et al. (2002) J. Bact. 184: 4177-4186; Skoble, et al. (2000) J. Cell Biol. 150: 527-538). In another embodiment, the Listeria strain is L. monocytogenes DP-L4042 (delta PEST) (see Brockstedt, et al. (2004) Proc. Natl. Acad. Sci. USA 101: 13832-13837; supporting information). In another embodiment, the Listeria strain is L. monocytogenes DP-L4097 (LLO-S44A) (see Brockstedt, et al. (2004) Proc. Natl. Acad. Sci. USA 101: 13832-13837; supporting information). In another embodiment, the Listeria strain is L. monocytogenes DP-L4364 (delta IplA; lipoate protein ligase) (see Brockstedt, et al. (2004) Proc. Natl. Acad. Sci. USA 101: 13832-13837; supporting information). In another embodiment, the Listeria strain is L. monocytogenes DP-L4405 (delta inlA) (see Brockstedt, et al. (2004) Proc. Natl. Acad. Sci. USA 101: 13832-13837; supporting information). In another embodiment, the Listeria strain is L. monocytogenes DP-L4406 (delta inlB) (see Brockstedt, et al. (2004) Proc. Natl. Acad. Sci. USA 101: 13832-13837; supporting information). In another embodiment, the Listeria strain is L. monocytogenes CS-L0001 (delta ActA-delta inlB) (see Brockstedt, et al. (2004) Proc. Natl. Acad. Sci. USA 101: 13832-13837; supporting information). In another embodiment, the Listeria strain is L. monocytogenes CS-L0002 (delta ActA-delta IplA) (see Brockstedt, et al. (2004) Proc. Natl. Acad. Sci. USA 101: 13832-13837; supporting information). In another embodiment, the Listeria strain is L. monocytogenes CS-L0003 (L461T-delta IplA) (see Brockstedt, et al. (2004) Proc. Natl. Acad. Sci. USA 101: 13832-13837; supporting information). In another embodiment, the Listeria strain is L. monocytogenes DP-L4038 (delta ActA-LLO L461T) (see Brockstedt, et al. (2004) Proc. Natl. Acad. Sci. USA 101: 13832-13837; supporting information). In another embodiment, the Listeria strain is L. monocytogenes DP-L4384 (S44A-LLO L461T) (see Brockstedt, et al. (2004) Proc. Natl. Acad. Sci. USA 101: 13832-13837; supporting information). In another embodiment, the Listeria strain is L. monocytogenes. Mutation in lipoate protein (see O'Riordan, et al. (2003) Science 302: 462-464). In another embodiment, the Listeria strain is L. monocytogenes DP-L4017 (10403S hly (L461T), having a point mutation in hemolysin gene (see U.S. Provisional Pat. Appl. Ser. No. 60/490,089 filed Jul. 24, 2003). In another embodiment, the Listeria strain is L. monocytogenes EGD (see GenBank Acc. No. AL591824). In another embodiment, the Listeria strain is L. monocytogenes EGD-e (see GenBank Acc. No. NC_003210. ATCC Acc. No. BAA-679). In another embodiment, the Listeria strain is L. monocytogenes DP-L4029 deleted in uvrAB (see U.S. Provisional Pat. Appl. Ser. No. 60/541,515 filed Feb. 2, 2004; U.S. Provisional Pat. Appl. Ser. No. 60/490,080 filed Jul. 24, 2003). In another embodiment, the Listeria strain is L. monocytogenes ActA-/inlB—double mutant (see ATCC Acc. No. PTA-5562). In another embodiment, the Listeria strain is L. monocytogenes IplA mutant or hly mutant (see U.S. Pat. Applic. No. 20040013690 of Portnoy, et. al). In another embodiment, the Listeria strain is L. monocytogenes DAL/DAT double mutant. (see U.S. Pat. Pub. No. 2005/0048081 of Frankel and Portnoy. In another embodiment, the Listeria strain is an L. monocytogenes dal/dat/actA mutant (see US Pat. Pub. 2011/0142791). The present disclosure encompasses reagents and methods that comprise the above Listerial strains, as well as these strains that are modified, e.g., by a plasmid and/or by genomic integration, to contain a nucleic acid encoding one of, or any combination of, the following genes: hly (LLO; listeriolysin); iap (p60); inlA; inlB; inlC; dal (alanine racemase); dat (D-amino acid aminotransferase); plcA; plcB; actA; or any nucleic acid that mediates growth, spread, breakdown of a single walled vesicle, breakdown of a double walled vesicle, binding to a host cell, uptake by a host cell. The present disclosure is not to be limited by the particular strains disclosed above.

In another embodiment, a recombinant Listeria strain of the present disclosure has been passaged through an animal host.

In another embodiment, the Listeria strain contains a genomic insertion of the gene encoding the antigen-containing recombinant peptide. In another embodiment, the Listeria strain carries a plasmid comprising the gene encoding the antigen-containing recombinant peptide.

In another embodiment, the recombinant Listeria strain utilized in methods and uses of the present disclosure has been stored in a frozen cell bank. In another embodiment, the recombinant Listeria strain has been stored in a lyophilized cell bank.

In another embodiment, the cell bank of methods and compositions of the present disclosure is a master cell bank. In another embodiment, the cell bank is a working cell bank. In another embodiment, the cell bank is Good Manufacturing Practice (GMP) cell bank. In another embodiment, the cell bank is intended for production of clinical-grade material. In another embodiment, the cell bank conforms to regulatory practices for human use. In another embodiment, the cell bank is any other type of cell bank known in the art.

“Good Manufacturing Practices” are defined, in another embodiment, by (21 CFR 210-211) of the United States Code of Federal Regulations. In another embodiment, “Good Manufacturing Practices” are defined by other standards for production of clinical-grade material or for human consumption; e.g. standards of a country other than the United States.

In another embodiment, a recombinant Listeria strain utilized in methods and uses of the present disclosure is from a batch of vaccine doses.

In another embodiment, a recombinant Listeria strain utilized in methods and uses of the present disclosure is from a frozen or lyophilized stock produced by methods provided in U.S. Pat. No. 8,114,414, which is incorporated by reference herein.

In another embodiment, a peptide of the present disclosure is a fusion peptide. In another embodiment, “fusion peptide” refers to a peptide or polypeptide comprising 2 or more proteins linked together by peptide bonds or other chemical bonds. In another embodiment, the proteins are linked together directly by a peptide or other chemical bond. In another embodiment, the proteins are linked together with 1 or more AA (e.g. a “spacer”) between the 2 or more proteins.

In another embodiment, a vaccine of the present disclosure further comprises an adjuvant. The adjuvant utilized in methods and compositions of the present disclosure is, in another embodiment, a granulocyte/macrophage colony-stimulating factor (GM-CSF) protein. In another embodiment, the adjuvant comprises a GM-CSF protein. In another embodiment, the adjuvant is a nucleotide molecule encoding GM-CSF. In another embodiment, the adjuvant comprises a nucleotide molecule encoding GM-CSF. In another embodiment, the adjuvant is saponin QS21. In another embodiment, the adjuvant comprises saponin QS21. In another embodiment, the adjuvant is monophosphoryl lipid A. In another embodiment, the adjuvant comprises monophosphoryl lipid A. In another embodiment, the adjuvant is SBAS2. In another embodiment, the adjuvant comprises SBAS2. In another embodiment, the adjuvant is an unmethylated CpG-containing oligonucleotide. In another embodiment, the adjuvant comprises an unmethylated CpG-containing oligonucleotide. In another embodiment, the adjuvant is an immune-stimulating cytokine. In another embodiment, the adjuvant comprises an immune-stimulating cytokine. In another embodiment, the adjuvant is a nucleotide molecule encoding an immune-stimulating cytokine. In another embodiment, the adjuvant comprises a nucleotide molecule encoding an immune-stimulating cytokine. In another embodiment, the adjuvant is or comprises a quill glycoside. In another embodiment, the adjuvant is or comprises a bacterial mitogen. In another embodiment, the adjuvant is or comprises a bacterial toxin. In another embodiment, the adjuvant is or comprises any other adjuvant known in the art.

In one embodiment, the methods and uses provided herein further comprise the step of administering a STING pathway agonist. In another embodiment, the immune composition comprising a recombinant Listeria further comprises a STING pathway agonist. In another embodiment, the STING pathway agonist is an antibody or fragment thereof. In another embodiment, a STING pathway agonist is a small molecule. In another embodiment, the small molecule STING pathway agonist is 5,6-dimethylxanthenone-4-acetic acid (DMXAA), a cyclic dinucleotide, a 2′3′-cGAMP or a hydrolysis-resistant bisphosphothioate analog of 2′3′-cGAMP (2′3′-cG^{s}A^{s}MP), or a combination thereof. In another, the STING pathway agonist is administered in a separate immunogenic composition than the composition comprising a recombinant Listeria. In another embodiment, the STING agonist is administered at the same time as the recombinant Listeria provided herein. In another embodiment, the STING agonist is administered before, during, or after administration of a recombinant Listeria provided herein.

In another embodiment, a nucleotide of the present disclosure is operably linked to a promoter/regulatory sequence that drives expression of the encoded peptide in the Listeria strain. Promoter/regulatory sequences useful for driving constitutive expression of a gene are well known in the art and include, but are not limited to, for example, the P_{hlyA}, P_{ActA}, and p60 promoters of Listeria, the Streptococcus bac promoter, the Streptomyces griseus sgiA promoter, and the B. thuringiensis phaZ promoter. In another embodiment, inducible and tissue specific expression of the nucleic acid encoding a peptide of the present disclosure is accomplished by placing the nucleic acid encoding the peptide under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the disclosure includes the use of any promoter/regulatory sequence, which is either known or unknown, and which is capable of driving expression of the desired protein operably linked thereto.

In another embodiment, either a whole E7 protein or a fragment thereof is fused to a LLO protein, ActA protein, or PEST amino acid sequence-containing peptide to generate a recombinant peptide of methods and uses of the present disclosure. The E7 protein that is utilized (either whole or as the source of the fragments) has, in another embodiment, the sequence

MHGDTPTLHEYMLDLQPETTDLYCYEQLNDSSEEEDEIDGPAGQAEP DRAHYNIVTFCCKCDSTLRLCVQSTHVDIRTLEDLLMGTLGIVCPICSQKP (SEQ ID No: 25). In another embodiment, the E7 protein is a homologue of SEQ ID No: 25. In another embodiment, the E7 protein is a variant of SEQ ID No: 25. In another embodiment, the E7 protein is an isomer of SEQ ID No: 25. In another embodiment, the E7 protein is a fragment of SEQ ID No: 25. In another embodiment, the E7 protein is a fragment of a homologue of SEQ ID No: 25. In another embodiment, the E7 protein is a fragment of a variant of SEQ ID No: 25. In another embodiment, the E7 protein is a fragment of an isomer of SEQ ID No: 25.

In another embodiment, the sequence of the E6 protein is:

MHGPKATLQDIVLHLEPQNEIPVDLLCHEQLSDSEEENDEIDGVNHQH LPARRAEPQRHTMLCMCCKCEARIELVVESSADDLRAFQQLFLNTLSFVCPW CASQQ (SEQ ID No: 26). In another embodiment, the E6 protein is a homologue of SEQ ID No: 26. In another embodiment, the E6 protein is a variant of SEQ ID No: 26. In another embodiment, the E6 protein is an isomer of SEQ ID No: 26. In another embodiment, the E6 protein is a fragment of SEQ ID No: 26. In another embodiment, the E6 protein is a fragment of a homologue of SEQ ID No: 26. In another embodiment, the E6 protein is a fragment of a variant of SEQ ID No: 26. In another embodiment, the E6 protein is a fragment of an isomer of SEQ ID No: 26.

In another embodiment, either a whole E6 protein or a fragment thereof is fused to a LLO protein, ActA protein, or PEST amino acid sequence-containing peptide to generate a recombinant peptide of methods and uses of the present disclosure. The E6 protein that is utilized (either whole or as the source of the fragments) has, in another embodiment, the sequence

MHQKRTAMFQDPQERPRKLPQLCTELQTTIHDIILECVYCKQQLLRRE VYDFAFRDLCIVYRDGNPYAVCDKCLKFYSKISEYRHYCYSLYGTTLEQQYN KPLCDLLIRCINCQKPLCPEEKQRHLDKKQRFHNIRGRWTGRCMSCCRSSRTR RETQL (SEQ ID No: 27). In another embodiment, the E6 protein is a homologue of SEQ ID No: 27. In another embodiment, the E6 protein is a variant of SEQ ID No: 27. In another embodiment, the E6 protein is an isomer of SEQ ID No: 27. In another embodiment, the E6 protein is a fragment of SEQ ID No: 27. In another embodiment, the E6 protein is a fragment of a homologue of SEQ ID No: 27. In another embodiment, the E6 protein is a fragment of a variant of SEQ ID No: 27. In another embodiment, the E6 protein is a fragment of an isomer of SEQ ID No: 27.

In another embodiment, the sequence of the E6 protein is:

MARFEDPTRRPYKLPDLCTELNTSLQDIEITCVYCKTVLELTEVFEFAF KDLFVVYRDSIPHAACHKCIDFYSRIRELRHYSDSVYGDTLEKLTNTGLYNLL IRCLRCQKPLNPAEKLRHLNEKRRFHNIAGHYRGQCHSCCNRARQERLQRRR ETQV (SEQ ID No: 28). In another embodiment, the E6 protein is a homologue of SEQ ID No: 28. In another embodiment, the E6 protein is a variant of SEQ ID No: 28. In another embodiment, the E6 protein is an isomer of SEQ ID No: 28. In another embodiment, the E6 protein is a fragment of SEQ ID No: 28. In another embodiment, the E6 protein is a fragment of a homologue of SEQ ID No: 28. In another embodiment, the E6 protein is a fragment of a variant of SEQ ID No: 28. In another embodiment, the E6 protein is a fragment of an isomer of SEQ ID No: 28.

In another embodiment, “homology” refers to identity to a sequence provided herein of greater than 70%. In another embodiment, “homology” refers to identity to one of the sequences provided herein of greater than 64%. In another embodiment, “homology” refers to identity to one of the sequences provided herein of greater than 68%. In another embodiment, “homology” refers to identity to one of the sequences provided herein of greater than 72%. In another embodiment, “homology” refers to identity to one of one of the sequences provided herein of greater than 75%. In another embodiment, of the identity is greater than 78%, greater than 80%, greater than 82%, greater than 83%, greater than 85%, greater than 87%, greater than 88%, greater than 90%, greater than 92%, greater than 93%, greater than 95%, greater than 96%, greater than 97%, greater than 98%, greater than 99%. In another embodiment, the term refers to identity of 100%.

Protein and/or peptide homology for any AA sequence listed herein is determined, in one embodiment, by methods well described in the art, including immunoblot analysis, or via computer algorithm analysis of AA sequences, utilizing any of a number of software packages available, via established methods. Some of these packages include the FASTA, BLAST, MPsrch or Scanps packages, and employ, in other embodiments, the use of the Smith and Waterman algorithms, and/or global/local or BLOCKS alignments for analysis, for example.

In another embodiment, the LLO protein, ActA protein, or fragment thereof is attached to the antigen by chemical conjugation. In another embodiment, glutaraldehyde is used for the conjugation. In another embodiment, the conjugation is performed using any suitable method known in the art.

In another embodiment, fusion proteins of the present disclosure are prepared by any suitable method, including, for example, cloning and restriction of appropriate sequences or direct chemical synthesis by methods discussed below. In another embodiment, subsequences are cloned and the appropriate subsequences cleaved using appropriate restriction enzymes. The fragments are then ligated, in another embodiment, to produce the desired DNA sequence. In another embodiment, DNA encoding the fusion protein is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The insert is then ligated into a plasmid.

In another embodiment, the LLO protein, ActA protein, or fragment thereof and the antigen, or fragment thereof are conjugated by a means known to those of skill in the art. In another embodiment, the antigen, or fragment thereof is conjugated, either directly or through a linker (spacer), to the ActA protein or LLO protein. In another embodiment, the chimeric molecule is recombinantly expressed as a single-chain fusion protein.

In another embodiment, a fusion peptide of the present disclosure is synthesized using standard chemical peptide synthesis techniques. In another embodiment, the chimeric molecule is synthesized as a single contiguous polypeptide. In another embodiment, the LLO protein, ActA protein, or fragment thereof; and the antigen, or fragment thereof are synthesized separately, then fused by condensation of the amino terminus of one molecule with the carboxyl terminus of the other molecule, thereby forming a peptide bond. In another embodiment, the ActA protein or LLO protein and antigen are each condensed with one end of a peptide spacer molecule, thereby forming a contiguous fusion protein.

In another embodiment, the peptides and proteins of the present disclosure are prepared by solid-phase peptide synthesis (SPPS) as described by Stewart et al. in Solid Phase Peptide Synthesis, 2nd Edition, 1984, Pierce Chemical Company, Rockford, Ill.; or as described by Bodanszky and Bodanszky (The Practice of Peptide Synthesis, 1984, Springer-Verlag, New York). In another embodiment, a suitably protected AA residue is attached through its carboxyl group to a derivatized, insoluble polymeric support, such as cross-linked polystyrene or polyamide resin. “Suitably protected” refers to the presence of protecting groups on both the alpha-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial AA, and couple thereto of the carboxyl end of the next AA in the sequence of the desired peptide. This AA is also suitably protected. The carboxyl of the incoming AA can be activated to react with the N-terminus of the support-bound AA by formation into a reactive group such as formation into a carbodiimide, a symmetric acid anhydride or an “active ester” group such as hydroxybenzotriazole or pentafluorophenly esters.

In another embodiment, the present disclosure provides a kit comprising vaccine of the present disclosure, an applicator, and instructional material that describes use of the methods of the disclosure. Although model kits are described below, the contents of other useful kits will be apparent to the skilled artisan in light of the present disclosure.

The compositions of this disclosure, in another embodiment, are administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally. In another embodiment, the compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present disclosure, the active ingredient is formulated in a capsule. In accordance with this embodiment, the compositions of the present disclosure comprise, in addition to the active compound and the inert carrier or diluent, a hard gelating capsule.

In another embodiment, compositions are administered by intravenous, intra-arterial, or intra-muscular injection of a liquid preparation. Suitable liquid formulations include solutions, suspensions, dispersions, emulsions, oils and the like. In one embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

As used throughout, the terms “composition” and “immunogenic composition” are interchangeable having all the same meanings and qualities. The term “pharmaceutical composition” refers, in some embodiments, to a composition suitable for pharmaceutical use, for example, to administer to a subject in need.

In one embodiment, the term “immunogenic composition” may encompass the recombinant Listeria provided herein, an adjuvant, a STING pathway agonist, or any combination thereof. In one embodiment, an immunogenic composition comprises a recombinant Listeria provided herein. In another embodiment, an immunogenic composition comprises an adjuvant known in the art or as provided herein. In another embodiment, an immunogenic composition comprises a STING agonist known in the art or as provided herein. It is also to be understood that administration of such compositions improves maturation of immunity, enhance an immune response, or increase a T effector cell to regulatory T cell ratio.

In one embodiment, this disclosure provides methods of use which comprise administering a composition comprising the described Listeria strains.

In one embodiment, the term “pharmaceutical composition” encompasses a therapeutically effective amount of the active ingredient or ingredients including the Listeria strain, together with a pharmaceutically acceptable carrier or diluent. It is to be understood that the term a “therapeutically effective amount” refers to that amount which provides a therapeutic effect for a given condition and administration regimen.

It will be understood by the skilled artisan that the term “administering” encompasses bringing a subject in contact with a composition of the present disclosure. In one embodiment, administration can be accomplished in vitro, i.e. in a test tube, or in vivo, i.e. in cells or tissues of living organisms, for example humans. In one embodiment, the present disclosure encompasses administering the Listeria strains and compositions thereof of the present disclosure to a subject.

The terms “contacting” or “administering,” in one embodiment, refer to directly contacting a cell or tissue of a subject with a composition of the present disclosure. In another embodiment, the terms refer to indirectly contacting a cell or tissue of a subject with a composition of the present disclosure.

The term “about” as used herein means in quantitative terms plus or minus 5%, or in another embodiment plus or minus 10%, or in another embodiment plus or minus 15%, or in another embodiment plus or minus 20%.

### EXPERIMENTAL DETAILS SECTION

### Example 1

### LLO-Antigen Fusions Induce Anti-Tumor Immunity Materials and Experimental Methods (Examples 1-2)

### Cell Lines

The C57BL/6 syngeneic TC-1 tumor was immortalized with HPV-16 E6 and E7 and transformed with the c-Ha-ras oncogene. TC-1, provided by T. C. Wu (Johns Hopkins University School of Medicine, Baltimore, Md.) is a highly tumorigenic lung epithelial cell expressing low levels of with HPV-16 E6 and E7 and transformed with the c-Ha-ras oncogene. TC-1 was grown in RPMI 1640, 10% FCS, 2 mM L-glutamine, 100 U/ml penicillin, 100 μg/ml streptomycin, 100 μM nonessential amino acids, 1 mM sodium pyruvate, 50 micromolar (mcM) 2-ME, 400 microgram (mcg)/ml G418, and 10% National Collection Type Culture-109 medium at 37° with 10% CO₂. C3 is a mouse embryo cell from C57BL/6 mice immortalized with the complete genome of HPV 16 and transformed with pEJ-ras. EL-4/E7 is the thymoma EL-4 retrovirally transduced with E7.

L. monocytogenes Strains and Propagation

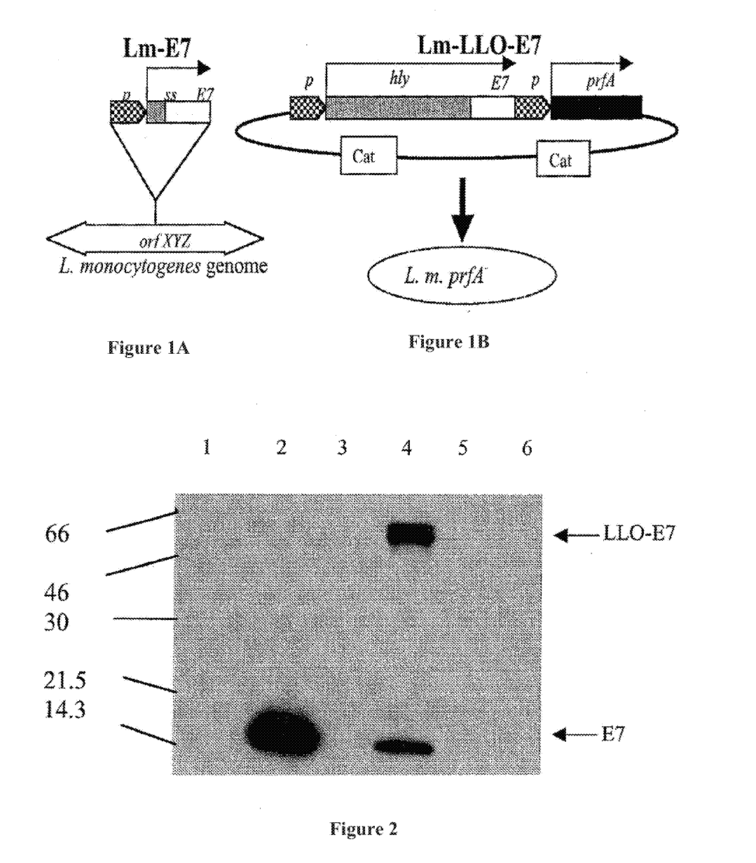

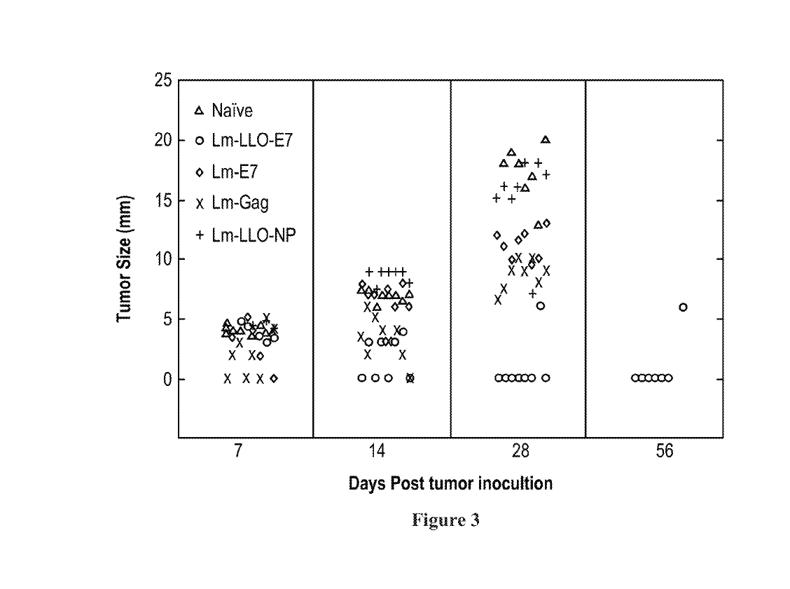

Listeria strains used were Lm-LLO-E7 (hly-E7 fusion gene in an episomal expression system; FIG. 1A), Lm-E7 (single-copy E7 gene cassette integrated into Listeria genome), Lm-LLO-NP (“DP-L2028”; hly-NP fusion gene in an episomal expression system), and Lm-Gag (“ZY-18”; single-copy HIV-1 Gag gene cassette integrated into the chromosome). E7 was amplified by PCR using the primers 5′-GGCTCGAGCATGGAGATACACC-3′ (SEQ ID No: 29; XhoI site is underlined) and 5′-GGGGACTAGTTTATGGTTTCTGAGAACA-3′ (SEQ ID No: 30; SpeI site is underlined) and ligated into pCR2.1 (Invitrogen, San Diego, Calif.). E7 was excised from pCR2.1 by XhoI/SpeI digestion and ligated into pGG-55. The hly-E7 fusion gene and the pluripotential transcription factor prfA were cloned into pAM401, a multicopy shuttle plasmid (Wirth R et al, J Bacteriol, 165: 831, 1986), generating pGG-55. The hly promoter drives the expression of the first 441 AA of the hly gene product, (lacking the hemolytic C-terminus, referred to below as “ΔLLO,” and having the sequence set forth in SEQ ID No: 24), which is joined by the XhoI site to the E7 gene, yielding a hly-E7 fusion gene that is transcribed and secreted as LLO-E7. Transformation of a prfA negative strain of Listeria, XFL-7 (provided by Dr. Hao Shen, University of Pennsylvania), with pGG-55 selected for the retention of the plasmid in vivo (FIGS. 1A-B). The hly promoter and gene fragment were generated using primers 5′-GGGGGCTAGCCCTCCTTTGATTAGTATATTC-3′ (SEQ ID No: 31; NheI site is underlined) and 5′-CTCCCTCGAGATCATAATTTACTTCATC-3′ (SEQ ID No: 32; XhoI site is underlined). The prfA gene was PCR amplified using primers 5′-GACTACAAGGACGATGACCGACAAGTGATAACCCGGGATCTAAATAAAT CCGTTT-3′ (SEQ ID No: 33; XbaI site is underlined) and 5′-CCCGTCGACCAGCTCTTCTTGGTGAAG-3′ (SEQ ID No: 34; SalI site is underlined). Lm-E7 was generated by introducing an expression cassette containing the hly promoter and signal sequence driving the expression and secretion of E7 into the orfZ domain of the LM genome. E7 was amplified by PCR using the primers 5′-GCGGATCCCATGGAGATACACCTAC-3′ (SEQ ID No: 35; BamHI site is underlined) and 5′-GCTCTAGATTATGGTTTCTGAG-3′ (SEQ ID No: 36; XbaI site is underlined). E7 was then ligated into the pZY-21 shuttle vector. LM strain 10403S was transformed with the resulting plasmid, pZY-21-E7, which includes an expression cassette inserted in the middle of a 1.6-kb sequence that corresponds to the orfX, Y, Z domain of the LM genome. The homology domain allows for insertion of the E7 gene cassette into the orfZ domain by homologous recombination. Clones were screened for integration of the E7 gene cassette into the orfZ domain. Bacteria were grown in brain heart infusion medium with (Lm-LLO-E7 and Lm-LLO-NP) or without (Lm-E7 and ZY-18) chloramphenicol (20 μg/ml). Bacteria were frozen in aliquots at −80° C. Expression was verified by Western blotting (FIG. 2).

### Western Blotting

Listeria strains were grown in Luria-Bertoni medium at 37° C. and were harvested at the same optical density measured at 600 nm. The supernatants were TCA precipitated and resuspended in 1× sample buffer supplemented with 0.1 N NaOH. Identical amounts of each cell pellet or each TCA-precipitated supernatant were loaded on 4-20% Tris-glycine SDS-PAGE gels (NOVEX, San Diego, Calif.). The gels were transferred to polyvinylidene difluoride and probed with an anti-E7 monoclonal antibody (mAb) (Zymed Laboratories, South San Francisco, Calif.), then incubated with HRP-conjugated anti-mouse secondary Ab (Amersham Pharmacia Biotech, Little Chalfont, U.K.), developed with Amersham ECL detection reagents, and exposed to Hyperfilm (Amersham Pharmacia Biotech).

### Measurement of Tumor Growth

Tumors were measured every other day with calipers spanning the shortest and longest surface diameters. The mean of these two measurements was plotted as the mean tumor diameter in millimeters against various time points. Mice were sacrificed when the tumor diameter reached 20 mm. Tumor measurements for each time point are shown only for surviving mice.

### Effects of Listeria Recombinants on Established Tumor Growth

Six- to 8-wk-old C57BL/6 mice (Charles River) received 2×10⁵ TC-1 cells s.c. on the left flank. One week following tumor inoculation, the tumors had reached a palpable size of 4-5 mm in diameter. Groups of eight mice were then treated with 0.1 LD₅₀ i.p. Lm-LLO-E7 (10⁷ CFU), Lm-E7 (10⁶ CFU), Lm-LLO-NP (10⁷ CFU), or Lm-Gag (5×10⁵ CFU) on days 7 and 14.

### ⁵¹ Cr Release Assay

C57BL/6 mice, 6-8 wk old, were immunized i.p. with 0.1 LD₅₀ Lm-LLO-E7, Lm-E7, Lm-LLO-NP, or Lm-Gag. Ten days post-immunization, spleens were harvested. Splenocytes were established in culture with irradiated TC-1 cells (100:1, splenocytes:TC-1) as feeder cells; stimulated in vitro for 5 days, then used in a standard ⁵¹Cr release assay, using the following targets: EL-4, EL-4/E7, or EL-4 pulsed with E7 H-2b peptide (RAHYNIVTF). E:T cell ratios, performed in triplicate, were 80:1, 40:1, 20:1, 10:1, 5:1, and 2.5:1. Following a 4-h incubation at 37° C., cells were pelleted, and 50 μl supernatant was removed from each well. Samples were assayed with a Wallac 1450 scintillation counter (Gaithersburg, Md.). The percent specific lysis was determined as [(experimental counts per minute (cpm)−spontaneous cpm)/(total cpm−spontaneous cpm)]×100.

### TC-1-Specific Proliferation

C57BL/6 mice were immunized with 0.1 LD₅₀ and boosted by i.p. injection 20 days later with 1 LD₅₀ Lm-LLO-E7, Lm-E7, Lm-LLO-NP, or Lm-Gag. Six days after boosting, spleens were harvested from immunized and naive mice. Splenocytes were established in culture at 5×10⁵/well in flat-bottom 96-well plates with 2.5×10⁴, 1.25×10⁴, 6×10³, or 3×10³ irradiated TC-1 cells/well as a source of E7 Ag, or without TC-1 cells or with 10 μg/ml Con A. Cells were pulsed 45 h later with 0.5 μCi [³H]thymidine/well. Plates were harvested 18 h later using a Tomtec harvester 96 (Orange, Conn.), and proliferation was assessed with a Wallac 1450 scintillation counter. The change in cpm was calculated as experimental cpm−no Ag cpm.

### Flow Cytometric Analysis

C57BL/6 mice were immunized intravenously (i.v.) with 0.1 LD₅₀ Lm-LLO-E7 or Lm-E7 and boosted 30 days later. Three-color flow cytometry for CD8 (53-6.7, PE conjugated), CD62 ligand (CD62L; MEL-14, APC conjugated), and E7 H-2Db tetramer was performed using a FACSCalibur® flow cytometer with CellQuest® software (Becton Dickinson, Mountain View, Calif.). Splenocytes harvested 5 days after the boost were stained at room temperature (rt) with H-2Db tetramers loaded with the E7 peptide (RAHYNIVTF) or a control (HIV-Gag) peptide. Tetramers were used at a 1/200 dilution and were provided by Dr. Larry R. Pease (Mayo Clinic, Rochester, Minn.) and by the NIAID Tetramer Core Facility and the NIH AIDS Research and Reference Reagent Program. Tetramer⁺, CD8⁺, CD62L^{low} cells were analyzed.

### B16F0-Ova Experiment

24 C57BL/6 mice were inoculated with 5×10⁵ B16F0-Ova cells. On days 3, 10 and 17, groups of 8 mice were immunized with 0.1 LD₅₀ Lm-OVA (10⁶ cfu), Lm-LLO-OVA (10⁸ cfu) and eight animals were left untreated.

### Statistics

For comparisons of tumor diameters, mean and SD of tumor size for each group were determined, and statistical significance was determined by Student's t test. p≦0.05 was considered significant.

### Results

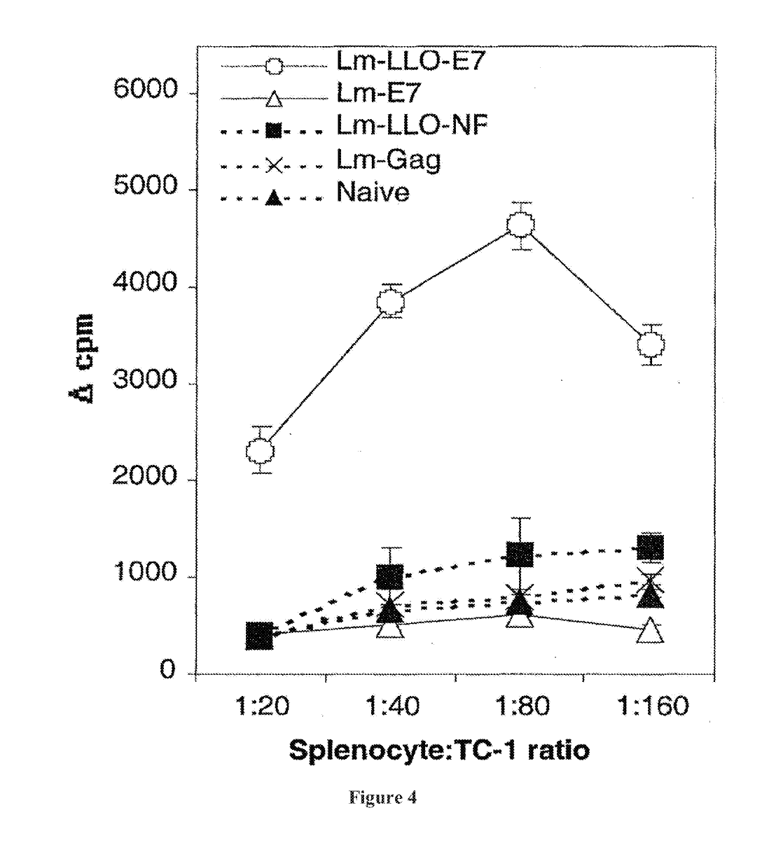

Lm-E7 and Lm-LLO-E7 were compared for their abilities to impact on TC-1 growth. Subcutaneous tumors were established on the left flank of C57BL/6 mice. Seven days later tumors had reached a palpable size (4-5 mm). Mice were vaccinated on days 7 and 14 with 0.1 LD₅₀ Lm-E7, Lm-LLO-E7, or, as controls, Lm-Gag and Lm-LLO-NP. Lm-LLO-E7 induced complete regression of 75% of established TC-1 tumors, while tumor growth was controlled in the other 2 mice in the group (FIG. 3). By contrast, immunization with Lm-E7 and Lm-Gag did not induce tumor regression. This experiment was repeated multiple times, always with very similar results. In addition, similar results were achieved for Lm-LLO-E7 under different immunization protocols. In another experiment, a single immunization was able to cure mice of established 5 mm TC-1 tumors.

In other experiments, similar results were obtained with 2 other E7-expressing tumor cell lines: C3 and EL-4/E7. To confirm the efficacy of vaccination with Lm-LLO-E7, animals that had eliminated their tumors were re-challenged with TC-1 or EL-4/E7 tumor cells on day 60 or day 40, respectively. Animals immunized with Lm-LLO-E7 remained tumor free until termination of the experiment (day 124 in the case of TC-1 and day 54 for EL-4/E7).

Thus, expression of an antigen as a fusion protein with ΔLLO enhances the immunogenicity of the antigen.

### Example 2

### Lm-LLO-E7 Treatment Elicits TC-1 Specific Splenocyte Proliferation

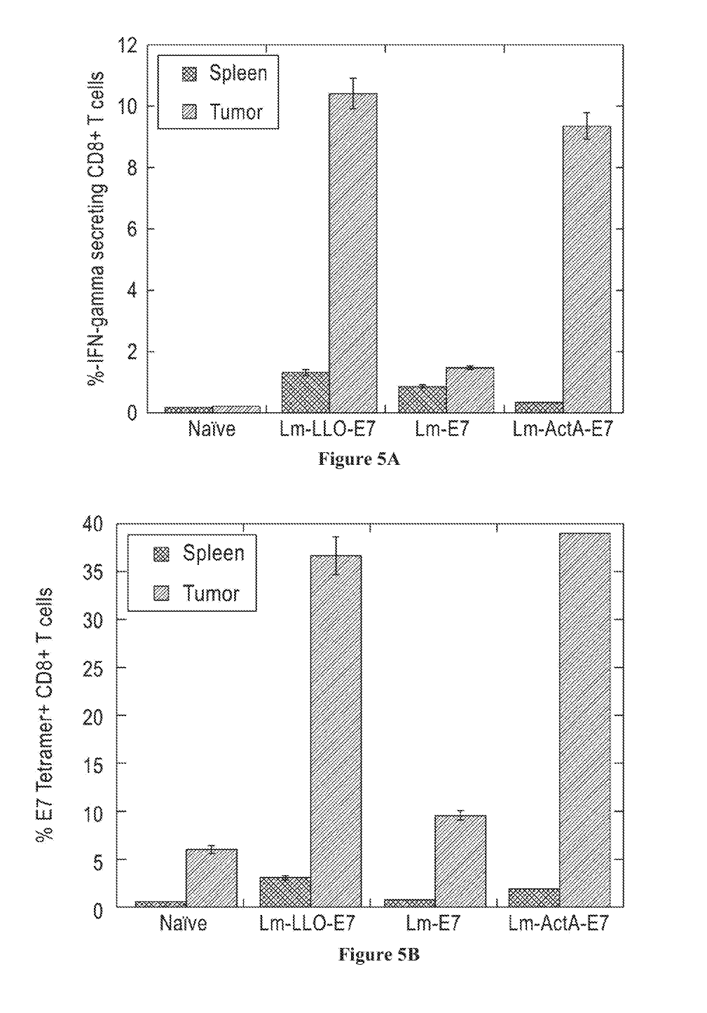

To measure induction of T cells by Lm-E7 with Lm-LLO-E7, TC-1-specific proliferative responses, a measure of antigen-specific immunocompetence, were measured in immunized mice. Splenocytes from Lm-LLO-E7-immunized mice proliferated when exposed to irradiated TC-1 cells as a source of E7, at splenocyte: TC-1 ratios of 20:1, 40:1, 80:1, and 160:1 (FIG. 4). Conversely, splenocytes from Lm-E7 and rLm control-immunized mice exhibited only background levels of proliferation.

### Example 3

### Fusion of E7 to LLO, ActA, or a Pest Amino Acid Sequence Enhances E7-Specific Immunity and Generates Tumor-Infiltrating E7-Specific CD8⁺ Cells

### Materials and Experimental Methods

500 mcl (microliter) of MATRIGEL®, comprising 100 mcl of 2×10⁵ TC-1 tumor cells in phosphate buffered saline (PBS) plus 400 mcl of MATRIGEL® (BD Biosciences, Franklin Lakes, N.J.) were implanted subcutaneously on the left flank of 12 C57BL/6 mice (n=3). Mice were immunized intraperitoneally on day 7, 14 and 21, and spleens and tumors were harvested on day 28. Tumor MATRIGELs were removed from the mice and incubated at 4° C. overnight in tubes containing 2 milliliters (ml) of RP 10 medium on ice. Tumors were minced with forceps, cut into 2 mm blocks, and incubated at 37° C. for 1 hour with 3 ml of enzyme mixture (0.2 mg/ml collagenase-P, 1 mg/ml DNAse-1 in PBS). The tissue suspension was filtered through nylon mesh and washed with 5% fetal bovine serum+0.05% of NaN₃ in PBS for tetramer and IFN-gamma staining.

Splenocytes and tumor cells were incubated with 1 micromole (mcm) E7 peptide for 5 hours in the presence of brefeldin A at 10⁷ cells/ml. Cells were washed twice and incubated in 50 mcl of anti-mouse Fc receptor supernatant (2.4 G2) for 1 hour or overnight at 4° C. Cells were stained for surface molecules CD8 and CD62L, permeabilized, fixed using the permeabilization kit Golgi-stop® or Golgi-Plug® (Pharmingen, San Diego, Calif.), and stained for IFN-gamma. 500,000 events were acquired using two-laser flow cytometer FACSCalibur and analyzed using Cellquest Software (Becton Dickinson, Franklin Lakes, N.J.). Percentages of IFN-gamma secreting cells within the activated (CD62L^{low}) CD8⁺ T cells were calculated.

For tetramer staining, H-2D^{b} tetramer was loaded with phycoerythrin (PE)-conjugated E7 peptide (RAHYNIVTF, SEQ ID NO: 37), stained at rt for 1 hour, and stained with anti-allophycocyanin (APC) conjugated MEL-14 (CD62L) and FITC-conjugated CD8□ at 4° C. for 30 min. Cells were analyzed comparing tetramer⁺CD8⁺ CD62L^{low} cells in the spleen and in the tumor.

### Results

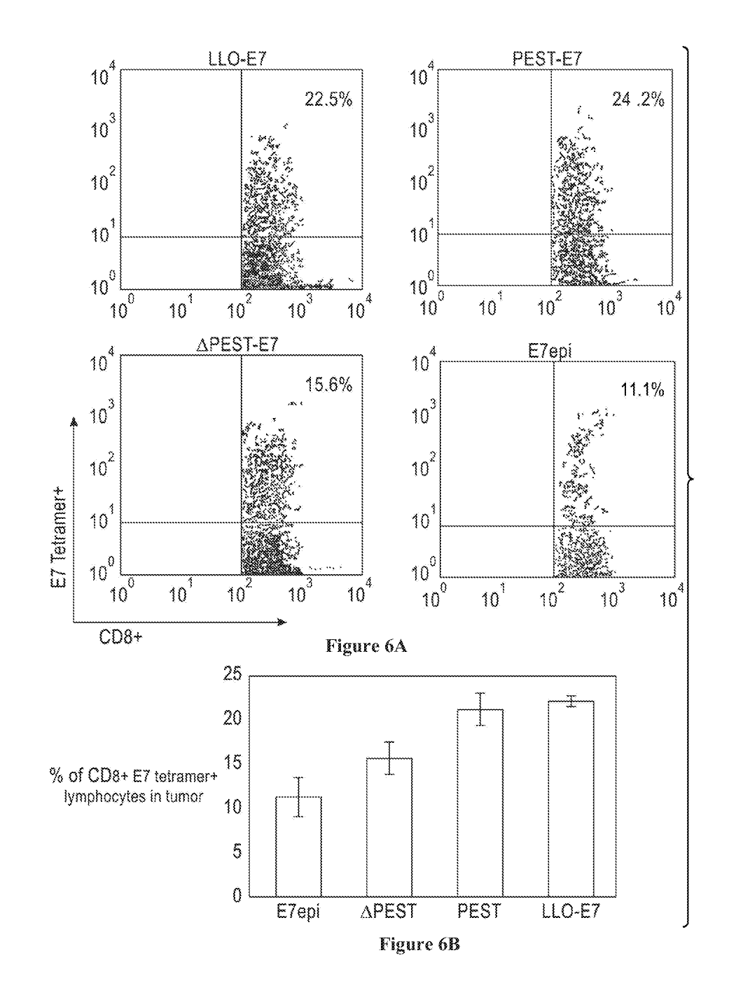

To analyze the ability of Lm-ActA-E7 to enhance antigen specific immunity, mice were implanted with TC-1 tumor cells and immunized with either Lm-LLO-E7 (1×10⁷ CFU), Lm-E7 (1×10⁶ CFU), or Lm-ActA-E7 (2×10⁸ CFU), or were untreated (naïve). Tumors of mice from the Lm-LLO-E7 and Lm-ActA-E7 groups contained a higher percentage of IFN-gamma-secreting CD8⁺ T cells (FIG. 5A) and tetramer-specific CD8⁺ cells (FIG. 5B) than in Lm-E7 or naive mice.

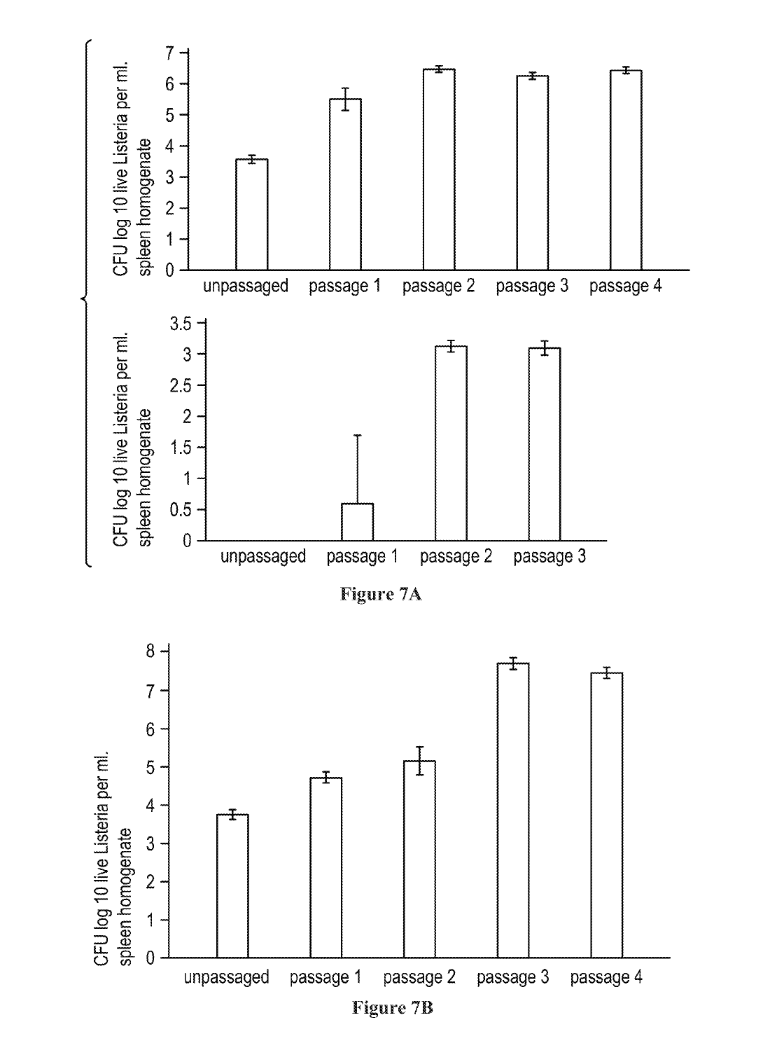

In another experiment, tumor-bearing mice were administered Lm-LLO-E7, Lm-PEST-E7, Lm-ΔPEST-E7, or Lm-E7epi, and levels of E7-specific lymphocytes within the tumor were measured. Mice were treated on days 7 and 14 with 0.1 LD₅₀ of the 4 vaccines. Tumors were harvested on day 21 and stained with antibodies to CD62L, CD8, and with the E7/Db tetramer. An increased percentage of tetramer-positive lymphocytes within the tumor were seen in mice vaccinated with Lm-LLO-E7 and Lm-PEST-E7 (FIG. 6A). This result was reproducible over three experiments (FIG. 6B).

Thus, Lm-LLO-E7, Lm-ActA-E7, and Lm-PEST-E7 are each efficacious at induction of tumor-infiltrating CD8⁺ T cells and tumor regression.

### Example 4

### Passaging of Listeria Vaccine Vectors Through Mice Elicits Increased Immune Responses to Heterologous and Endogenous Antigens

### Materials and Experimental Methods

### Bacterial Strains

L. monocytogenes strain 10403S, serotype 1 (ATCC, Manassas, Va.) was the wild type organism used in these studies and the parental strain of the constructs described below. Strain 10403S has an LD₅₀ of approximately 5×10⁴ CFU when injected intraperitoneally into BALB/c mice. “Lm-Gag” is a recombinant LM strain containing a copy of the HIV-1 strain HXB (subtype B laboratory strain with a syncytia-forming phenotype) gag gene stably integrated into the listerial chromosome using a modified shuttle vector pKSV7. Gag protein was expressed and secreted by the strain, as determined by Western blot. All strains were grown in brain-heart infusion (BHI) broth or agar plates (Difco Labs, Detroit, Mich.).

### Bacterial Culture

Bacteria from a single clone expressing the passenger antigen and/or fusion protein were selected and cultured in BHI broth overnight. Aliquots of this culture were frozen at ⁻70° C. with no additives. From this stock, cultures were grown to 0.1-0.2 O.D. at 600 nm, and aliquots were again frozen at −70° C. with no additives. To prepare cloned bacterial pools, the above procedure was used, but after each passage a number of bacterial clones were selected and checked for expression of the target antigen, as described herein. Clones in which expression of the foreign antigen was confirmed were used for the next passage.

### Passage of Bacteria in Mice

6-8 week old female BALB/c (H-2d) mice were purchased from Jackson Laboratories (Bar Harbor, Me.) and were maintained in a pathogen-free microisolator environment. The titer of viable bacteria in an aliquot of stock culture, stored frozen at −70° C., was determined by plating on BHI agar plates on thawing and prior to use. In all, 5×10⁵ bacteria were injected intravenously into BALB/c mice. After 3 days, spleens were harvested, homogenized, and serial dilutions of the spleen homogenate were incubated in BHI broth overnight and plated on BHI agar plates. For further passage, aliquots were again grown to 0.1-0.2 O.D., frozen at −70° C., and bacterial titer was again determined by serial dilution. After the initial passage (passage 0), this sequence was repeated for a total of 4 times.

### Intracellular Cytokine Stain for IFN-Gamma

Lymphocytes were cultured for 5 hours in complete RPMI-10 medium supplemented with 50 U/ml human recombinant IL-2 and 1 microliter/ml Brefeldin A (Golgistop™; PharMingen, San Diego, Calif.) in the presence or absence of either the cytotoxic T-cell (CTL) epitope for HIV-GAG (AMQMLKETI; SEQ ID No: 38), Listeria LLO (GYKDGNEYI; SEQ ID No: 39) or the HPV virus gene E7 (RAHYNIVTF (SEQ ID No: 40), at a concentration of 1 micromole. Cells were first surface-stained, then washed and subjected to intracellular cytokine stain using the Cytofix/Cytoperm kit in accordance with the manufacturer's recommendations (PharMingen, San Diego, Calif.). For intracellular IFN-gamma stain, FITC-conjugated rat anti-mouse IFN-gamma monoclonal antibody (clone XMG 1.2) and its isotype control Ab (rat IgG1; both from PharMingen) was used. In all, 10⁶ cells were stained in PBS containing 1% Bovine Serum Albumin and 0.02% sodium azide (FACS Buffer) for 30 minutes at 4° C. followed by 3 washes in FACS buffer. Sample data were acquired on either a FACScan™ flowcytometer or FACSCalibur™ instrument (Becton Dickinson, San Jose, Calif.). Three-color flow cytometry for CD8 (PERCP conjugated, rat anti-mouse, clone 53-6.7 Pharmingen, San Diego, Calif.), CD62L (APC conjugated, rat anti-mouse, clone MEL-14), and intracellular IFN-gamma was performed using a FACSCalibur™ flow cytometer, and data were further analyzed with CELLQuest software (Becton Dickinson, Mountain View, Calif.). Cells were gated on CD8 high and CD62L^{low} before they were analyzed for CD8⁺ and intracellular IFN-gamma staining.

### Results

### Passaging in Mice Increases the Virulence of Recombinant Listeria Monocytogenes

Three different constructs were used to determine the impact of passaging on recombinant Listeria vaccine vectors. Two of these constructs carry a genomic insertion of the passenger antigen: the first comprises the HIV gag gene (Lm-Gag), and the second comprises the HPV E7 gene (Lm-E7). The third (Lm-LLO-E7) comprises a plasmid with the fusion gene for the passenger antigen (HPV E7) fused with a truncated version of LLO and a gene encoding prfA, the positive regulatory factor that controls Listeria virulence factors. This plasmid was used to complement a prfA negative mutant so that in a live host, selection pressures would favor conservation of the plasmid, because without it the bacterium is avirulent. All 3 constructs had been propagated extensively in vitro for many bacterial generations.

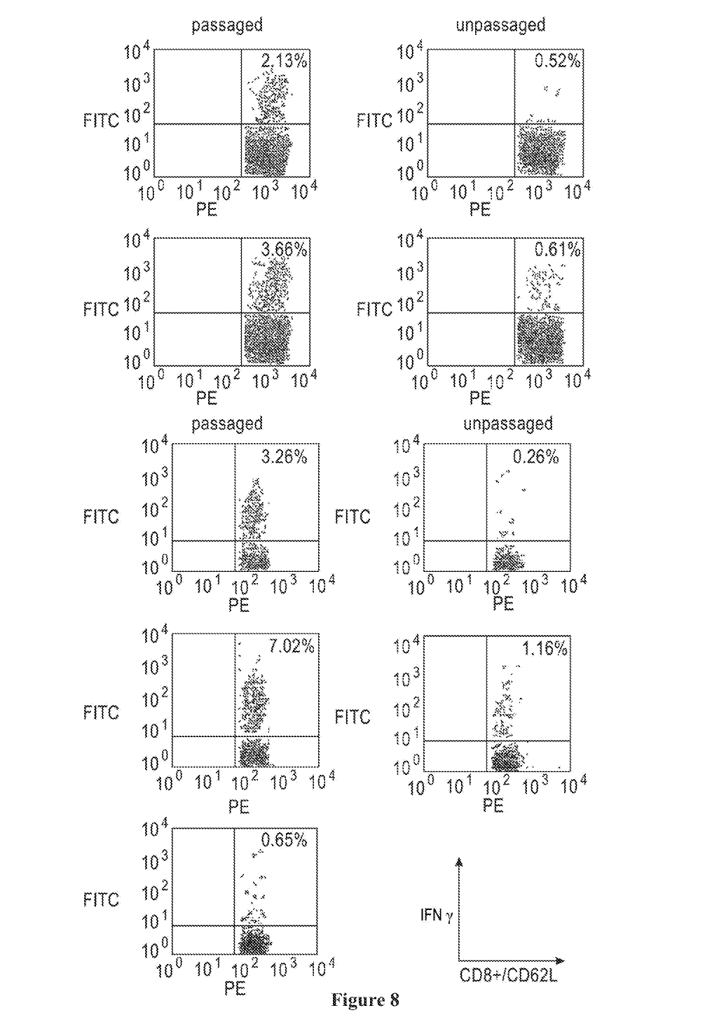

Passaging the bacteria resulted in an increase in bacterial virulence, as measured by numbers of surviving bacteria in the spleen, with each of the first 2 passages. For Lm-Gag and Lm-LLO-E7, virulence increased with each passage up to passage 2 (FIG. 7A). The plasmid-containing construct, Lm-LLO-E7, demonstrated the most dramatic increase in virulence. Prior to passage, the initial immunizing dose of Lm-LLO-E7 had to be increased to 10⁷ bacteria and the spleen had to be harvested on day 2 in order to recover bacteria (whereas an initial dose of 10⁵ bacteria for Lm-Gag was harvested on day 3). After the initial passage, the standard dosage of Lm-LLO-E7 was sufficient to allow harvesting on day 3. For Lm-E7, virulence increased by 1.5 orders of magnitude over unpassaged bacteria (FIG. 7B).

Thus, passage through mice increases the virulence of Listeria vaccine strains.

Passaging Increases the Ability of L. monocytogenes to Induce CD8⁺ T Cells

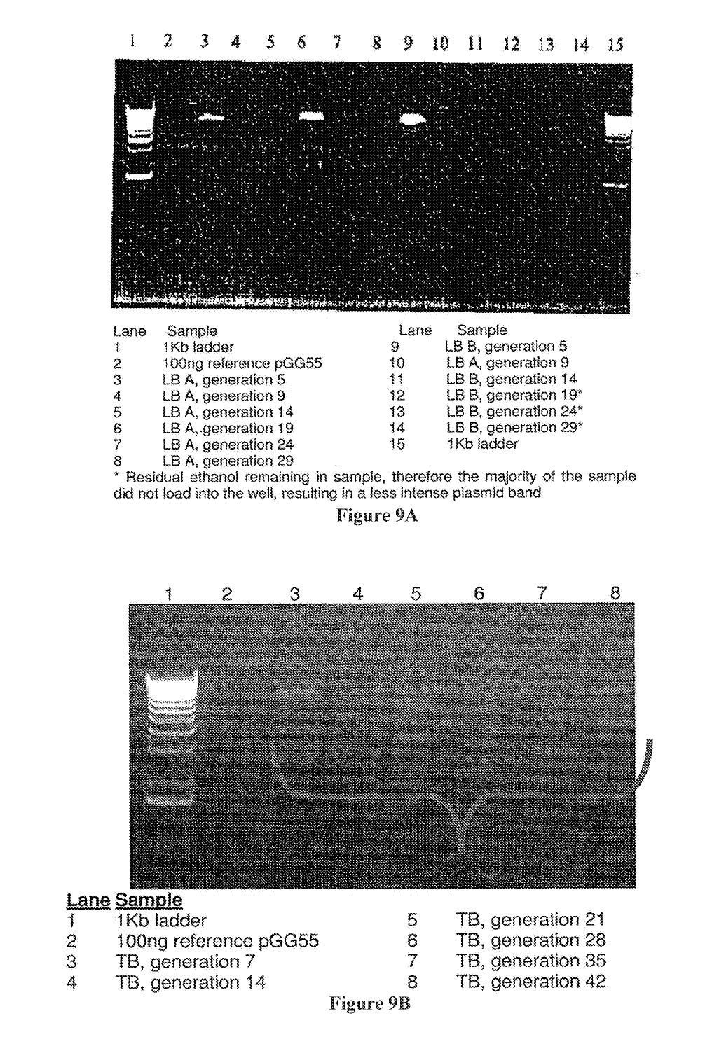

Next, the effect of passaging on induction of antigen-specific CD8⁺ T cells was determined by intracellular cytokine staining with immunodominant peptides specific for MHC-class I using HIV-Gag peptide AMQMLKETI (SEQ ID No: 41) and LLO 91-99 (GYKDGNEYI; SEQ ID No: 41). Injection of 10³ CFU passaged bacteria (Lm-Gag) into mice elicited significant numbers of HIV-Gag-specific CD8⁺ T cells, while the same dose of non-passaged Lm-Gag induced no detectable Gag-specific CD8⁺ T cells. Even increasing the dose of unpassaged bacteria 100-fold did not compensate for their relative avirulence; in fact, no detectable Gag-specific CD8⁺ T cells were elicited even at the higher dose. The same dose increase with passaged bacteria increased Gag-specific T cell induction by 50% (FIG. 8). The same pattern of induction of antigen-specific CD8⁺ T cells was observed with LLO-specific CD8⁺ T cells, showing that these results were not caused by the properties of the passenger antigen, since they were observed with LLO, an endogenous Listeria antigen.

Thus, passage through mice increases the immunogenicity of Listeria vaccine strains.

### Example 5

### A prfA-Containing Plasmid is Stable in an Lm Strain with a prfA Deletion in the Absence of Antibiotics

### Materials and Experimental Methods

### Bacteria

L. monocytogenes strain XFL7 contains a 300 base pair deletion in the prfA gene XFL7 carries pGG55 which partially restores virulence and confers CAP resistance, and is described in United States Patent Application Publication No. 200500118184.

Development of Protocol for Plasmid Extraction from Listeria

1 mL of Listeria monocytogenes Lm-LLO-E7 research working cell bank vial was inoculated into 27 mL BH1 medium containing 34 μg/mL CAP and grown for 24 hours at 37° C. and 200 rpm.

Seven 2.5 mL samples of the culture were pelleted (15000 rpm for 5 minutes), and pellets were incubated at 37° C. with 50 μl lysozyme solution for varying amounts of time, from 0-60 minutes.

Lysozyme Solution:
29 μl 1 M dibasic Potassium Phosphate
21 μl 1 M monobasic Potassium Phosphate
500 μl 40% Sucrose (filter sterilized through 0.45/μm filter)
450 μl water
60 μl lysozyme (50 mg/mL)

After incubation with the lysozyme, the suspensions were centrifuged as before and the supernatants discarded. Each pellet was then subjected to plasmid extraction by a modified version of the QIAprep Spin Miniprep Kit® (Qiagen, Germantown, Md.) protocol. The changes to the protocol were as follows:
1. The volumes of buffers PI, P2 and N3 were all increased threefold to allow complete lysis of the increased biomass.
2. 2 mg/mL of lysozyme was added to the resuspended cells before the addition of P2. The lysis solution was then incubated at 37° C. for 15 minutes before neutralization.
3. The plasmid DNA was resuspended in 30 μL rather than 50 μL to increase the concentration.

In other experiments, the cells were incubated for 15 min in P1 buffer+Lysozyme, then incubated with P2 (lysis buffer) and P3 (neutraliztion buffer) at room temperature.

Equal volumes of the isolated plasmid DNA from each subculture were run on an 0.8% agarose gel stained with ethidium bromide and visualized for any signs of structural or segregation instability.

The results showed that plasmid extraction from L. monocytogenes Lm-LLO-E7 increases in efficiency with increasing incubation time with lysozyme, up to an optimum level at approximately 50 minutes incubation.

These results provide an effective method for plasmid extraction from Listeria vaccine strains.

### Replica Plating

Dilutions of the original culture were plated onto plates containing LB or TB agar in the absence or presence of 34 μg/mL CAP. The differences between the counts on selective and non-selective agar were used to determine whether there was any gross segregational instability of the plasmid.

### Results

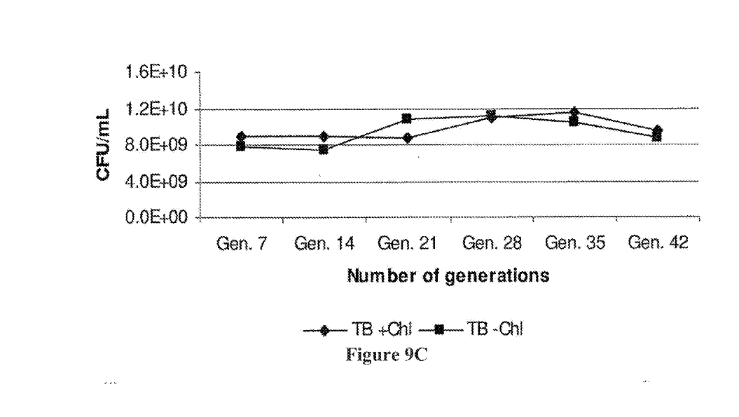

The genetic stability (i.e. the extent to which the plasmid is retained by or remains stably associated with the bacteria in the absence of selection pressure; e.g. antibiotic selection pressure) of the pGG55 plasmid in L. monocytogenes strain XFL7 in the absence of antibiotic was assessed by serial sub-culture in both Luria-Bertani media (LB: 5 g/L NaCl, 10 g/ml soy peptone, 5 g/L yeast extract) and Terrific Broth media (TB: 10 g/L glucose, 11.8 g/L soy peptone, 23.6 g/L yeast extract, 2.2 g/L KH₂PO₄, 9.4 g/L K₂HPO₄), in duplicate cultures. 50 mL of fresh media in a 250 mL baffled shake flask was inoculated with a fixed number of cells (1 ODmL), which was then subcultured at 24 hour intervals. Cultures were incubated in an orbital shaker at 37° C. and 200 rpm. At each subculture the OD₆₀₀ was measured and used to calculate the cell doubling time (or generation) elapsed, until 30 generations were reached in LB and 42 in TB. A known number of cells (15 ODmL) at each subculture stage (approximately every 4 generations) were pelleted by centrifugation, and the plasmid DNA was extracted using the Qiagen QIAprep Spin Miniprep® protocol described above. After purification, plasmid DNA was subjected to agarose gel electrophoresis, followed by ethidium bromide staining. While the amount of plasmid in the preps varied slightly between samples, the overall trend was a constant amount of plasmid with respect to the generational number of the bacteria (FIGS. 9A-B). Thus, pGG55 exhibited stability in strain XFL7, even in the absence of antibiotic.

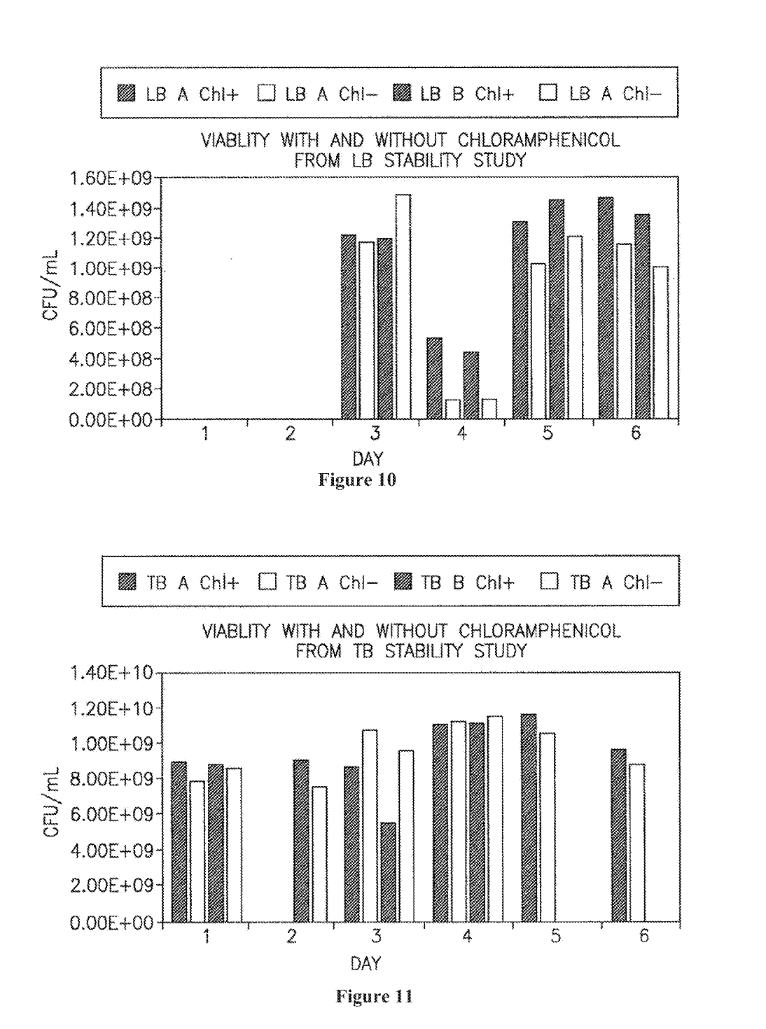

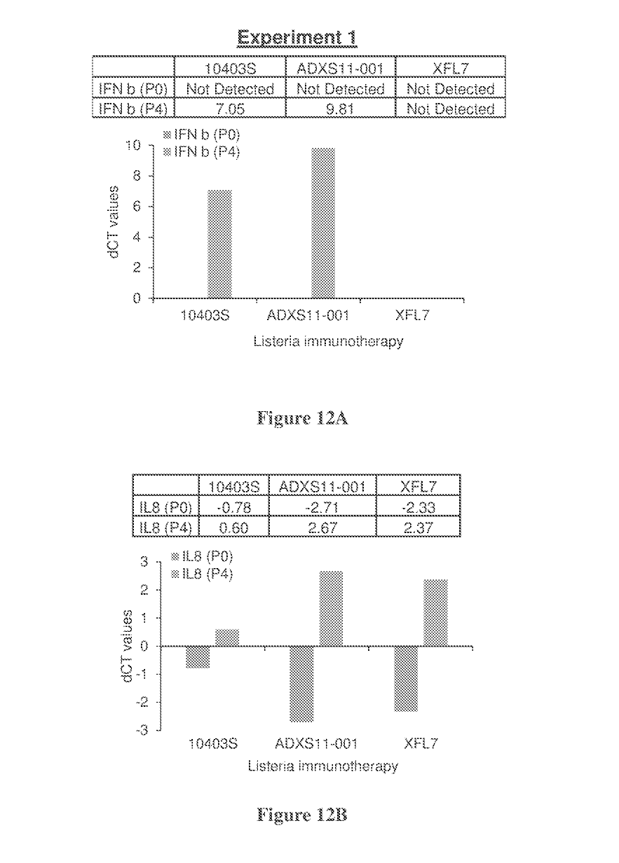

Plasmid stability was also monitored during the stability study by replica plating on agar plates at each stage of the subculture. Consistent with the results from the agarose gel electrophoresis, there was no overall change in the number of plasmid-containing cells throughout the study in either LB or TB liquid culture (FIGS. 10 and 11, respectively).

These findings demonstrate that prfA-encoding plasmids exhibit stability in the absence of antibiotic in Listeria strains containing mutations in prfA.

### Example 6

### Listeria-Mediated Activation of Sting Pathway

### Materials and Methods

Materials:

RPMI 1640, fetal bovine Serum, Phorbol 12-myristate 13-acetate (PMA), β-mercaptoethanol, Gentamicin, THP-1 cells and TRI reagent were purchased from. L-Glutamine was from Corning.

Methods:

Complete Medium (c-RPMI)

Complete medium was prepared by mixing 450 ml of RPMI 1640, 50 ml of fetal calf serum (FCS), 5 ml of 100+ L-Glutamine, and 129 ul of 14.6M 2-Mercaptoethanol

THP-1 Cell Culture

THP-1 cells were counted and suspended in pre-warmed c-RPMI with PMA (16 nM final conc/well), to 1×10⁶ cell/ml. 1 ml of 1×10⁶ cell/ml suspension was distributed per well in a 24 well plate. Cells were incubated at 37° C., 5% CO2 overnight.

Vaccine Preparation:

The next day, one frozen vial of 10403S, XFL-7 and ADXS11-001 (Lm-LLO-E7) was thawed at 37° C. water bath. The bacterial culture was centrifuged at 14,000 rpm for 2 min. Supernatant was discarded and the bacteria was washed two times with 1 ml of PBS. The culture was re-suspended in complete RPMI to a final concentration as shown below:

TABLE 1

| Construct | Final concentration |
|---|---|
| 10403S-WT | 1 × 10⁶/mL |
| ADXS11-001 | 1 × 10⁷/mL |
| XFL-7 | 1 × 10⁷/mL |

Infection Assay:

THP-1 cells adhere to the surface post PMA treatment. Media was removed from 24 well plate and 1 ml of C-RPMI with Lm constructs were added to wells. THP-1 cells were infected with either 10403S (MOI 1:1), XFL7 (MOI 10:1) or ADXS11-001 (MOI 10:1). The cells were incubated at 37° C., 5% CO₂ for 2 h. Post incubation, medium was discarded and 1 ml of c-RPMI with 50 μg/ml Gentamycin was added to kill all the Lm that was not taken up by the THP-1 cells. Cells were incubated in presence of Gentamicin for 45 min at 37° C., 5% CO₂. At the end of incubation medium was discarded and the cells were washed with 1 ml PBS and re-suspended in 1 ml of c-RPMI and incubated at 37° C., 5% CO₂, 5 min. For time P0, 1 ml of TRI reagent was added to the well and the cells were collected by pipetting up and down in an RNAse/DNAse free Eppendorf tube. For time P4, cells infected with Lm was incubated in c-RPMI for 4 hours post infection and at the end of 4 hours, cells were collected by pipetting up and down in an RNAse/DNAse free Eppendorf tube.

### RNA Preparation:

RNA was isolated from the samples using TRI reagent as per the manufacturer's instructions. All samples were treated with DNAse I to get rid of residual DNAse if present in the samples. Changes in expression of IFN beta and control gene (IL8) were studied with Real Time PCR using gene specific Taqman primers from Life Technologies (see Table 2). Glyceraldehyde-3-phosphate dehydrogenase (GAPDH) was used as internal control.

TABLE 2

| Primer | Sequence |
|---|---|
| human GAPDH-F | GCCGCATCTTCTTTTGCGTC |
| | (SEQ ID No: 43) |
| human GAPDH-R | TCGCCCCACTTGATTTTGGA |
| | (SEQ ID No: 44) |
| human IFN-G-F | ATGGTTGTCCTGCCTGCAAT |
| | (SEQ ID No: 45) |
| human IFN-G-R | CTTGCTTAGGTTGGCTGCCT |
| | (SEQ ID No: 46) |
| human IL-8-F | AGTCCTTGTTCCACTGTGCC |
| | (SEQ ID No: 47) |
| human IL-8-R | CACAGCACTACCAACACAGC |
| | (SEQ ID No: 48) |

### Results

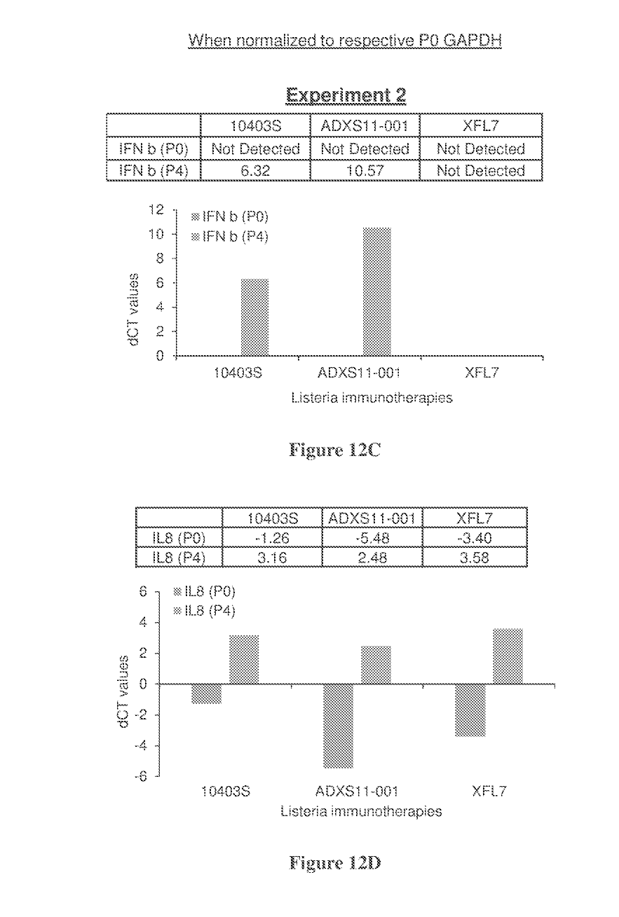

THP-1 cells, a human monocytic cell line, were used to evaluate if our immunotherapy, ADXS11-001 was able to activate STING pathway. Increased production of IFNβ, a type I IFN was used as a readout of STING activation. THP-1 cells are often used for the study of DNA sensing pathways as they express all the cytosolic DNA sensors identified so far. THP-1 are maintained in monocytic state but can easily be differentiated into macrophage phenotype using PMA stimulation. PMA stimulated THP-1 cells were infected with 10403S (wild type Listeria), XFL7 (mutant Listeria that fails to escape phagolysosome) and ADXS11-001 (Lm-LLO-E7) and changes in expression of IFN-beta were monitored. It was observed that IFN-β was induced at 4 hours post infection only for 10403S and ADXS11-001 and not for XFL7 (FIG. 12A-B).

Having described preferred embodiments of the disclosure with reference to the accompanying drawings, it is to be understood that the disclosure is not limited to the precise embodiments, and that various changes and modifications may be effected therein by those skilled in the art without departing from the scope or spirit of the disclosure as defined in the appended claims.

What is claimed is:

1. A method of activating and enhancing a STimulator of INterferon Genes (STING) complex pathway in a host cell in a subject having a tumor or cancer, the method comprising the step of administering to said subject a composition comprising a recombinant Listeria strain capable of expressing a hemolytic LLO protein from a genomic LLO gene, wherein said activation and enhancement of said STING pathway enhances an immune response in said subject, thereby activating and enhancing a STING pathway.

2. The method of claim 1, wherein said Listeria strain comprises multiple copies of a recombinant double stranded nucleic acid, said nucleic acid comprising a first open reading frame encoding a recombinant polypeptide comprising an N-terminal fragment of an LLO protein, wherein said recombinant nucleic acid further comprises a second open reading frame encoding a mutant prfA gene or a metabolic enzyme, wherein administering said Listeria induces an anti-tumor or an anti-cancer immune response in said subject.

3. The method of claim 1, wherein said LLO protein is fused to a first heterologous antigen or fragment thereof.

4. The method of any one of claims 1-3, wherein activating and enhancing said STING pathway leads to an enhanced production of interferons.

5. The method of claim 4, wherein said interferon is IFN-beta.

6. The method of any one of claims 2-5, wherein said mutant prfA gene contains a D133V mutation.

7. The method of any one of claims 2-6, wherein said mutant prfA gene complements a prfA genomic mutation or deletion.

8. The method of any one of claims 1-7, wherein said administering is intravenous or oral administering.

9. The method of any one of claims 2-8, wherein said N-terminal fragment of an LLO protein comprises SEQ ID NO: 2.

10. The method of any one of claims 1-9, wherein said subject is human.

11. The method of any one of claims 1-10, wherein said recombinant Listeria strain is administered to said human subject at a dose of 1×10⁹-3.31×10¹⁰ organisms.

12. The method of any one of claims 1-11, wherein said recombinant Listeria strain is a recombinant Listeria monocytogenes strain.

13. The method of any one of claims 1-12, wherein said recombinant Listeria strain has been passaged through an animal host, prior to the step of administering.

14. The method of any one of claims 2-13, wherein said recombinant polypeptide is expressed by said recombinant Listeria strain.

15. The method of any one of claims 2-14, wherein said recombinant Listeria strain comprises a multi-copy plasmid that encodes said recombinant polypeptide.

16. The method of claim 15, wherein said plasmid is an extrachromosomal plasmid that is stably maintained in the recombinant Listeria strain in the absence of antibiotic selection.

17. The method of claim 15, wherein said plasmid is an integrative plasmid comprising sequences for integration into the Listeria chromosome.

18. The method of any one of claims 1-17, wherein said recombinant nucleic acid is a double-stranded nucleic acid.

19. The method of any one of claims 1-18, wherein said Listeria strain comprises a mutation or inactivation in the genomic dal, dat, and actA genes.

20. The method of any one of claims 2-19, wherein said metabolic enzyme complements a mutation in the gene encoding D-alanine racemase enzyme or in the gene encoding D-amino acid transferase enzyme.

21. The method of any one of claims 2-19, wherein said metabolic enzyme complements a mutation in the gene encoding a D-alanine racemase enzyme and in the gene encoding a D-amino acid transferase enzyme.

22. The method of any one of claims 2-21, wherein said metabolic enzyme is a D-alanine racemase enzyme or a D-amino acid transferase enzyme.

23. The method of any one of claims 2-22, wherein said recombinant nucleic acid in said Listeria comprises a third open reading frame encoding a second heterologous antigen or a functional fragment thereof individually fused to an N-terminal LLO protein fragment.

24. The method of claim 23, wherein said recombinant nucleic acid in said Listeria comprises a fourth open reading frame encoding a third heterologous antigen or a functional fragment thereof individually fused to an N-terminal LLO protein fragment.

25. The method of any one of claims 3, 23, and 24, further comprising the step of inoculating said human subject with an immunogenic composition that comprises or directs expression of said heterologous antigen.

26. The method of any one of claims 1-25, further comprising administering a STING pathway agonist.

27. The method of claim 26, wherein said agonist is an antibody or fragment thereof, or a small molecule.

28. The method of claim 27, wherein said small molecule is 5,6-dimethylxanthenone-4-acetic acid (DMXAA), a cyclic dinucleotide or a combination thereof.

29. The method of any one of claims 1-28, wherein said method allows for an enhanced expression of IFN-beta leading to a potent anti-tumor cytotoxic T cell response.

30. The method of any one of claims 1-28, wherein said method comprises protecting said subject against a tumor or cancer.

31. The method of any one of claims 1-28, wherein said method induces an anti-tumor cytotoxic T cell response in said subject.

32. The method of any one of claims 1-28, wherein said method comprises treating a subject having a tumor or cancer.

33. The method of any one of claims 1-28, wherein said immune response reduces the need of said subject having said tumor or said cancer to receive chemotherapeutic or radiation treatment.

34. The method of any one of claims 1-28, wherein said immune response reduces the severity of side effects associated with a follow-up radiation or chemotherapeutic treatment in said subject.

35. The method of any one of claims 1-28, wherein said immune response eliminates the need of a follow-up radiation or chemotherapeutic treatment in said subject having said tumor or cancer.

36. The method of any one of claims 29-35, further comprising the step of administering a booster dose of said composition comprising said recombinant Listeria strain to said subject.
